(19) United States
(12) Patent Application Publication
(10) Pub. No.: US 2023/0013430 A1
(43) Pub. Date: Jan. 19, 2023

(54) SULFONIUM SALT, ACID GENERATOR, RESIST COMPOSITION, AND METHOD FOR PRODUCING DEVICE

(71) Applicant: TOYO GOSEI CO., LTD., Ichikawa-shi, Chiba (JP)

(72) Inventors: Yusuke SUGA, Ichikawa-shi (JP); Yoshiyuki UTSUMI, Ichikawa-shi (JP)

(73) Assignee: TOYO GOSEI CO., LTD., Ichikawa-shi, Chiba (JP)

(21) Appl. No.: 17/771,960

(22) PCT Filed: Sep. 16, 2020
(86) PCT No.: PCT/JP2020/035151

§ 371 Date: Apr. 26, 2022

(30) Foreign Application Priority Data
Nov. 12, 2019 (JP) 2019-205012

(51) Int. Cl. G03F 7/004; C07C 381/12; C07C 309/06; C07C 309/12; C07D 333/76; C07C 65/10

(52) U.S. Cl. CPC G03F 7/0045; C07C 381/12; C07C 309/06; C07C 309/12; C07D 333/76; C07C 65/10; C07C 2603/74

(57) ABSTRACT

A sulfonium salt represented by a following general formula (1),

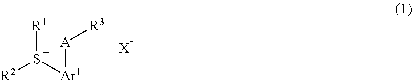

wherein in the general formula (1), each of R¹ to R³ is independently an alkyl group, an aryl group, or a heteroaryl group;

at least one carbon-carbon single bond contained in the alkyl group is optionally substituted with a carbon-carbon double bond or a carbon-carbon triple bond;

at least one methylene group contained in the alkyl group is optionally substituted with at least one divalent heteroatom-containing group;

Ar¹ is an arylene group;

at least one of R¹, R², R³ and Ar¹ has at least one substituent (R);

at least two of R¹ to R³, Ar¹, and substituent (R) optionally form a ring;

A is a divalent group selected from a group consisting of —S—, —SO—, and —SO₂—;

Ar¹ is substituted with A at an ortho-position with respect to a sulfonio group (S⁺); and

X⁻ is an anion.

### TECHNICAL FIELD

Some aspects of the present invention relate to a sulfonium salt useful as an acid generator for a chemically amplified photoresist composition. Also, some aspects of the present invention relate to an acid generator that decomposes by irradiation of an active energy beam to generate an acid, a resist composition containing the acid generator, and a method of manufacturing a device using the resist composition.

### BACKGROUND ART

In the field of semiconductor devices, for example, highly integrated circuit elements such as DRAM, there is a high demand for higher density, higher integration, or higher speed. Along with this, in the field of manufacturing various electronic devices, the establishment of half-micron order microfabrication technology, for example, the development of photolithography technology for forming fine patterns is increasingly required. In order to form fine patterns in photolithography technology, it is necessary to improve the resolution. Here, a resolution (R) of a reduced projection exposure system is defined by Rayleigh's equation R=k·λ/NA, wherein λ is a wavelength of an active energy beam, NA is a numerical aperture of a lens, and k is a process factor. The resolution can be improved by shortening the wavelength λ of the active energy beam used for forming the resist pattern.

As a short wavelength active energy beam, KrF excimer laser (248 nm), ArF excimer laser (193 nm), EUV (extreme ultraviolet radiation, 13.5 nm), and an electron beam tend to be used. The lithography techniques using an active energy beam, particularly EUV or an electron beam, enable microfabrication with single patterning. Therefore, it is considered that the need for a resist composition having high sensitivity to EUV or an electron beam will be further increased in the future.

A chemically amplified photoresist has been proposed as a photoresist suitable for use with a short wavelength active energy beam. The chemically amplified photoresist has a characteristic that an acid is generated from an acid generator contained in the chemically amplified photoresist by irradiation with an active energy beam and this acid causes an acid catalytic reaction by heat treatment after exposure.

It is known that in a chemically amplified resist using a short wavelength active energy beam, there is a trade-off relationship between sensitivity, resolution and LWR (Line Width Roughness), and it is difficult to improve these lithographic performance at the same time.

As an acid generator used in chemically amplified photoresists, a sulfonium salt having a (4-phenylsulfanylphenyl) diphenylsulfonium skeleton is known (Patent Literature 1).

However, the photoresist composition using the sulfonium salt having the above skeleton has not sufficiently satisfied the lithographic performance such as sensitivity, resolution and LWR.

### CITATION LIST

### Patent Literature

PATENT LITERATURE 1: JP2008-120700

### SUMMARY OF INVENTION

### Technical Problem

In view of these circumstances, some aspects of the present invention provide a sulfonium salt having excellent lithographic performance such as sensitivity, resolution, and LWR. They also provide an acid generator using the sulfonium salt, a resist composition containing the acid generator, and a method for manufacturing a device using the resist composition.

### Solution to Problem

As a result of diligent studies to solve the above problem, the inventor has found that, by using a sulfonium salt having a specific structure as an acid generator in a resist composition, the sensitivity of the resist composition is enhanced, and furthermore, the resist composition has excellent lithographic properties such as resolution and LWR, completing some aspects of the present invention.

One aspect of the present invention that solves the above problem is a sulfonium salt represented by the following general formula (1).

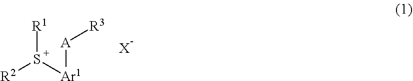

In the above general formula (1), each of R¹ to R³ is independently an alkyl group having 1 to 30 carbon atoms, an aryl group having 6 to 30 carbon atoms, or a heteroaryl group having 4 to 30 carbon atoms. At least one carbon-carbon single bond contained in the above alkyl group is optionally substituted with a carbon-carbon double bond or a carbon-carbon triple bond. Ar¹ is an arylene group having 6 to 30 carbon atoms. At least one of R¹, R², R³, and Ar¹ has at least any substituent (R) selected from a group consisting of a monovalent organic group having 1 to 20 carbon atoms, an amino group, a hydroxyl group, a cyano group, a nitro group, and a halogen atom. At least two of R¹ to R³, Ar', and the above substituent (R) optionally form a ring together with the sulfur atom to which R¹ to R³ or Ar¹ is bonded and/or A, directly with a single bond or via any divalent group selected from a group consisting of an oxygen atom, a sulfur atom, a nitrogen atom-containing group, and an alkylene group. A is a divalent group selected from a group consisting of —S—, —SO—, and —SO₂—. Ar¹ is substituted with the A at an ortho-position with respect to the sulfonio group (S⁺). X⁻ is an anion.

Another aspect of the present invention is an acid generator containing the above sulfonium salt.

Another aspect of the present invention is a resist composition containing the above acid generator and an acid reactive compound.

Another aspect of the present invention is a method for manufacturing a device, including: a resist film formation step of applying the above resist composition on a substrate to form a resist film; a photolithography step of exposing the resist film to an active energy beam; and a patterning step of developing the exposed resist film to obtain a photoresist pattern.

### Effect of the Invention

The sulfonium salt according to one aspect of the present invention is useful as an acid generator that efficiently generates an acid by irradiation with an active energy beam. In addition, when the sulfonium salt according to one aspect of the present invention is used as an acid generator in a resist composition, the sensitivity of the resist composition is enhanced, and the lithographic properties such as resolution and LWR of the resist composition tend to be excellent.

### DESCRIPTION OF EMBODIMENTS

The following is a detailed description of the invention.

### <1> Sulfonium Salt

The sulfonium salt according to one aspect of the present invention is represented by the above general formula (1).

Each of R¹ to R³ of the above general formula (1) is independently an alkyl group having 1 to 30 carbon atoms, an aryl group having 6 to 30 carbon atoms, or a heteroaryl group having 4 to 30 carbon atoms.

Examples of the above alkyl group of R¹ to R³ include: linear alkyl groups such as a methyl group, an ethyl group, an n-propyl group and an n-butyl group; branched alkyl groups such as an isopropyl group and a t-butyl group; and cyclic alkyl groups such as a cyclopropyl group, a cyclobutyl group, a cyclopentyl group, a cyclohexyl group, an adamantyl group and a norbornyl group; and the like.

At least one carbon-carbon single bond contained in the above alkyl groups of R¹ to R³ is optionally substituted with a carbon-carbon double bond or a carbon-carbon triple bond. Examples of such an alkyl group include: double bond-containing alkyl groups such as a vinyl group, an allyl group and a homoallyl group (—CH₂—CH₂—CH═CH₂); and triple bond-containing alkyl groups such as a propargyl group and a homopropargyl (—CH₂—CH₂—C≡CH); and the like.

At least one methylene group contained in the above alkyl group is optionally substituted with at least any divalent heteroatom-containing group selected from a group consisting of —O—, —CO—, —NH—, —S—, —SO— and —SO₂—.

The upper limit of the number of carbon atoms of the above alkyl group is preferably 20, and more preferably 10.

Examples of the above aryl group of R¹ to R³ include: monovalent monocyclic aromatic hydrocarbon groups such as a phenyl group; monovalent condensed polycyclic aromatic hydrocarbon groups such as a naphthyl group, an anthryl group, a phenanthrenyl group, a pentarenyl group, an indenyl group, an indacenyl group, an acenaphthyl group, a fluorenyl group, and a heptalenyl group, a naphthacenyl group, a pyrenyl group and a chrysenyl group; and monovalent linked polycyclic aromatic hydrocarbon groups such as a biphenyl group, a terphenyl group and a quaterphenyl group; and the like.

The upper limit of the number of carbon atoms of the above aryl group is preferably 20, and more preferably 10.

Examples of the above heteroaryl group of R¹ to R³ include monovalent groups obtained by removing one hydrogen atom from heterocycles such as: monocyclic aromatic heterocycles such as furan, thiophene, pyrrole, imidazole, pyrazole, oxazole, pyridine, pyran, pyrimidine, and pyrazine; condensed polycyclic aromatic heterocycles such as indole, purine, quinoline, isoquinoline, chromen, thianthrene, dibenzothiophene, phenothiazine, phenoxazine, xanthene, acridine, phenazine and carbazole; and linked polycyclic aromatic heterocycles such as 4-phenylpyridine, 9-phenylacridine and bathophenanthroline; and the like.

The upper limit of the number of carbon atoms of the above heteroaryl group is preferably 20, and more preferably 10.

Ar¹ in the above general formula (1) is an arylene group having 6 to 30 carbon atoms. Examples of the arylene group include a divalent group obtained by removing one hydrogen atom from the above aryl groups of R¹ to R³.

The upper limit of the number of carbon atoms of the above arylene group is preferably 20, and more preferably 10.

At least one of R¹, R², R³ and Ar¹ has at least any substituent (R) selected from the group consisting of a monovalent organic group having 1 to 20 carbon atoms, an amino group, a hydroxyl group, a cyano group, a nitro group and a halogen atom. The upper limit of the number of carbon atoms of the above monovalent organic group is preferably 15, and more preferably 10.

As the monovalent organic group of the substituent (R), a monovalent group represented by the following general formula (3) can be mentioned.

*—(Ls)ₙₛ—Q (3)

In the above general formula (3), * represents a bond to R¹, R², R³ or Ar¹ in the above general formula (1). When there are a plurality of Ls, each of them is independently selected from a group consisting of a single bond, —O—, —CO—, —NH—, —NR^{s}, —NAr—, —NAr^{h}—, —S—, —SO—, —SO₂—, an alkylene group having 1 to 20 carbon atoms, an arylene group having 6 to 20 carbon atoms and a heteroarylene group having 4 to 20 carbon atoms. At least one methylene group contained in the alkylene group having 1 to 20 carbon atoms is optionally substituted with at least any divalent heteroatom-containing group selected from a group consisting of —O—, —CO—, —NH—, —S—, —SO— and —SO₂—. Q is selected from a group consisting of —R^{s}, —Ar and —Ar^{h}. ns is an integer of 0 to 10. The upper limit of ns is preferably 5, and more preferably 3.

Each of R^{s} in Q and Ls represents an alkyl group having 1 to 20 carbon atoms. Examples of the alkyl group include the same groups as the above alkyl groups of R¹ to R³. The upper limit of the number of carbon atoms of the alkyl group is preferably 15, and more preferably 10. Each of Ar in the Q and Ls represents an aryl group having 6 to 20 carbon atoms. Examples of the aryl group include the same groups as the above aryl groups of R¹ to R³. The upper limit of the number of carbon atoms of the aryl group is preferably 15, and more preferably 10. Each of Ar^{h} in the Q and Ls represents a heteroaryl group having 4 to 20 carbon atoms. Examples of the heteroaryl group include the same groups as the above heteroaryl groups of R¹ to R³. The upper limit of the number of carbon atoms of the heteroaryl group is preferably 15, and more preferably 10.

Examples of the alkylene group having 1 to 20 carbon atoms in Ls include a group obtained by removing one hydrogen atom from the alkyl group in R^{s}. The upper limit of the number of carbon atoms of the alkylene group is preferably 15, and more preferably 10. Examples of the above arylene group having 6 to 20 carbon atoms in the Ls include a group obtained by removing one hydrogen atom from the aryl group in Ar. The upper limit of the number of carbon atoms of the arylene group is preferably 15, and more preferably 10. Examples of the heteroarylene group having 4 to 20 carbon atoms in Ls include a group obtained by removing one hydrogen atom from the heteroaryl group in Ar^{h}. The upper limit of the number of carbon atoms of the heteroarylene group is preferably 15, and more preferably 10.

Examples of the above monovalent group represented by the above general formula (3) include: linear alkyl groups such as a methyl group, an ethyl group, an n-propyl group and an n-butyl group; branched alkyl groups such as an isopropyl and a t-butyl group; cyclic alkyl groups such as a cyclopropyl group, a cyclobutyl group, a cyclopentyl group, a cyclohexyl group, an adamantyl group and norbornyl group; double bond-containing alkyl groups such as a vinyl group, an allyl group, a homoallyl group and an isopropenyl group; triple bond-containing alkyl groups such as a propargyl group and a homopropargyl group; monocyclic aromatic hydrocarbon groups such as a phenyl group; condensed polycyclic aromatic hydrocarbon groups such as an α-naphthyl group and a β-naphthyl group; monocyclic aromatic heterocyclic groups such as a furanyl group, a thienyl group and a pyridyl group; condensed polycyclic aromatic heterocyclic groups such as an indolyl group, a quinolinyl group and a xanthenyl group; arylalkyl groups such as a benzyl group and a 2-phenylethyl group; heteroarylalkyl groups such as a pyridylmethyl group and a thienylmethyl group; arylheteroaryl groups such as a 4-phenylpyridyl group and a 9-phenylacridinyl group; alkoxy groups such as a methoxy group, an ethoxy group, a propoxy group and a butoxy group; aryloxy groups such as a phenoxy group, an α-naphthoxy group and a β-naphthoxy group; polyether groups such as a methoxymethyloxy group and a phenoxymethyloxy group; alkylcarbonyl groups such as an acetyl group, an ethylcarbonyl group and a propylcarbonyl group; arylcarbonyl groups such as a benzoyl group, an α-naphthylcarbonyl group and a β-naphthylcarbonyl group; heteroarylcarbonyl groups such as a furylcarbonyl, a pyridylcarbonyl, and a thienylcarbonyl group; alkylcarbonyloxy groups such as an acetyloxy group, an ethylcarbonyloxy group and a propylcarbonyloxy group; arylcarbonyloxy groups such as a benzoyloxy group, an α-naphthylcarbonyloxy group and a β-naphthylcarbonyloxy group; heteroarylcarbonyloxy groups such as a furylcarbonyloxy group, a pyridylcarbonyloxy group and a thienylcarbonyloxy group; alkoxycarbonyl groups such as a methoxycarbonyl group, an ethoxycarbonyl group, a propoxycarbonyl group and a butoxycarbonyl group; aryloxycarbonyl groups such as a phenoxycarbonyl group, an α-naphthoxycarbonyl group and a β-naphthoxycarbonyl; alkylamino groups such as a methylamino group, a dimethylamino group and a dibutylamino group; arylamino groups such as a phenylamino group and a diphenylamino group; alkylarylamino groups such as an N-methyl-N-phenylamino group and an N-butyl-N-phenylamino group; alkylaminocarbonyl groups such as a methylaminocarbonyl group, a butylaminocarbonyl and a dimethylaminocarbonyl group; arylaminocarbonyl groups such as a phenylaminocarbonyl group and a diphenylaminocarbonyl group; alkylarylaminocarbonyl groups such as an N-methyl-N-phenylaminocarbonyl group and an N-butyl-N-phenylaminocarbonyl group; acylamino groups such as an acetylamino group, a benzoylamino group, an N-acetyl-N-methylamino group, an N-benzoyl-N-methylamino group, an N-acetyl-N-phenylamino group, and an N-benzoyl-N-phenylamino group.

Examples of the monovalent group represented by the above general formula (3) include radically polymerizable groups such as a vinyl group, an allyl group, an isopropenyl group, an acryloxy group and a methacryloxy group. When the monovalent organic group is the above radically polymerizable group, the acid generator according to one aspect of the present invention can be used as a polymer component. When the acid generator is used as a polymer component, the diffusion of the generated acid is suppressed, and the resolution and LWR tend to be improved.

When the acid generator according to one aspect of the present invention is used as a polymer component, the number of carbon atoms of the above monovalent organic group in the above sulfonium salt represents a number of carbon atoms of a linking group from each of the above R¹ to R³ and Ar¹ to the main chain of the polymer. That is, a number of carbon atoms of the structural unit other than the above sulfonium salt and a number of carbon atoms of the main chain of the polymer are not included in the number of carbon atoms of the monovalent organic group.

When the substituent (R) is the above monovalent organic group, the above monovalent organic group may further have a substituent (hereinafter, also referred to as “substituent (r)”). The number of carbon atoms of R¹ to R³ and Ar¹ is a number of carbon atoms including the number of carbon atoms of the substituent (R) and the number of carbon atoms of the substituent (r).

Examples of the above substituent (r) include: an amino group; a hydroxyl group; a cyano group; a nitro group; a halogen atom; an halogenated alkyl group; a halogenated aryl group; and a group having an onium structure such as a sulfonium, an iodonium, an ammonium and a phosphonium.

Examples of the above substituent (R) having the substituent (r) include: amino group-containing groups such as an aminomethyl group and an aminophenyl group; hydroxyl group-containing groups such as a hydroxymethyl group and a hydroxyphenyl group; cyano group-containing groups such as a cyanomethyl group and a cyanophenyl group; nitro group-containing groups such as a nitromethyl group and a nitrophenyl group; halogenated alkyl groups such as a trifluoromethyl group and a trichloromethyl group; and halogenated aryl groups such as a 4-trifluoromethylphenyl group and a perfluorophenyl group.

Examples of the halogen atom of the substituent (R) include a fluorine atom, a chlorine atom, a bromine atom and an iodine atom.

The above substituent (R) is preferably an alkyl group, an alkoxy group, a cyano group, a nitro group, an amino group, an alkylamino group, an acyloxy group, an alkyloxycarbonyl group, a halogen atom, and an halogenated alkyl group from the viewpoint of sensitivity to an active energy beam. As the above substituent (R), halogen atoms such as a fluorine atom and halogenated alkyl groups such as a trifluoromethyl group are particularly preferable.

At least two of R¹ to R³, Ar¹, and the above substituent (R) optionally form a ring together with the sulfur atom to which R¹ to R³ or Ar¹ is bonded and/or A, directly via a single bond or via any divalent group selected from a group consisting of an oxygen atom, a sulfur atom, a nitrogen atom-containing group, and an alkylene group.

Examples of the nitrogen atom-containing group include divalent groups containing a nitrogen atom such as an aminodiyl group (—NH—), an alkylaminodiyl group (—NR^{s}—) and an arylaminodiyl group (—NAr—). In addition, each of R^{s} and Ar is selected from the same groups as the above alkyl group and the above aryl group in the above substituent (R).

Examples of the alkylene group include a group obtained by removing one hydrogen atom from the alkyl groups of R¹ to R³ of the above general formula (1). The alkylene group preferably has 1 to 30 carbon atoms. The upper limit of the number of carbon atoms of the alkylene group is preferably 20, more preferably 10, and even more preferably 5. At least one methylene group contained in the alkylene group may be substituted with at least any divalent heteroatom-containing group selected from a group consisting of —O—, —CO—, —NH—, —S—, —SO— and —SO₂—.

The A is a divalent group selected from the group consisting of —S—, —SO—, and —SO₂—.

Ar¹ is substituted with the A at an ortho-position with respect to the sulfonio group (S⁺). Since Ar¹ is substituted with the A at the ortho-position with respect to the sulfonio group (S⁺) and at least one of R¹, R², R³, and Ar¹ has the substituent (R), the sulfonium salt efficiently absorbs an active energy beam to efficiently generate an acid. Therefore, the resist composition containing the sulfonium salt as an acid generator tends to have high sensitivity, and the lithographic properties such as resolution and LWR tend to be improved.

In Ar¹, the “ortho-position with respect to the sulfonio group” represents a substitution position adjacent to the sulfonio group. For example, when Ar¹ is a naphthalene ring, the sulfonio group and the A being at any of the 1,2-positions, 2,3-positions, 3,4-positions, 5,6-positions, 6,7-positions and 7,8-positions are referred to the substitution with the A at the ortho-position with respect to the sulfonio group.

X⁻ is an anion.

In the above general formula (1), the following general formulas can be exemplified as examples of the above sulfonium salt. In the following compounds, A, R, and X⁻ are the same as A, the substituent (R), and X⁻ in the above general formula (1).

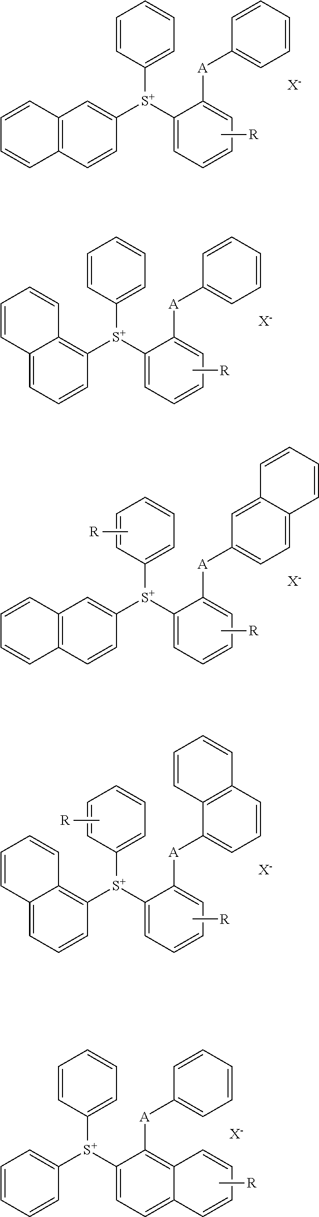

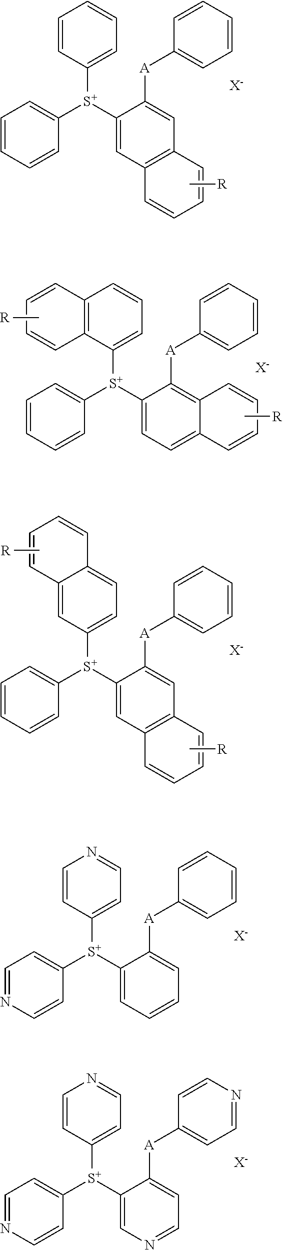

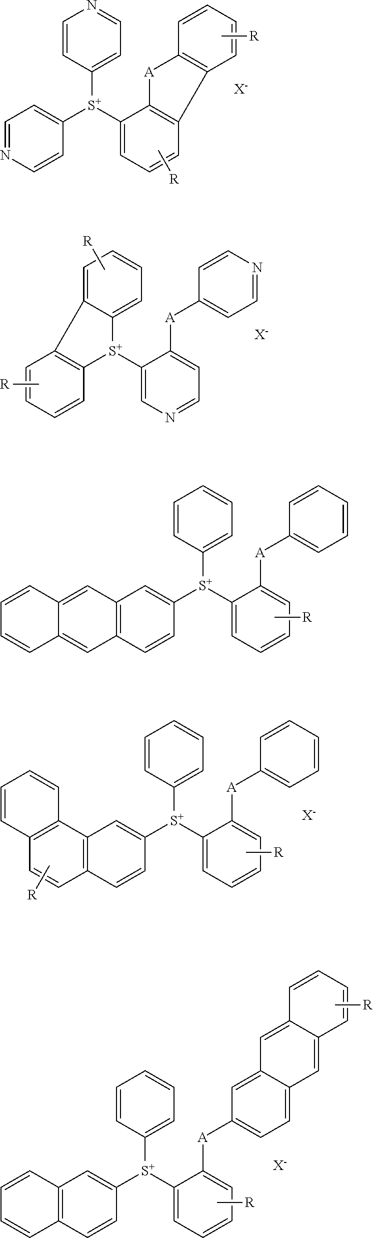

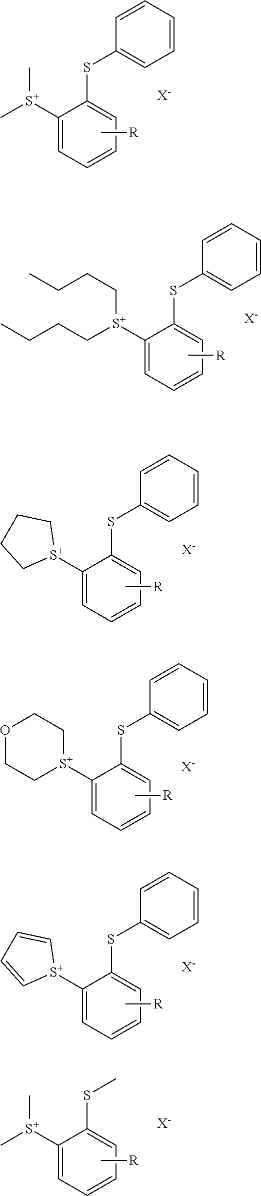

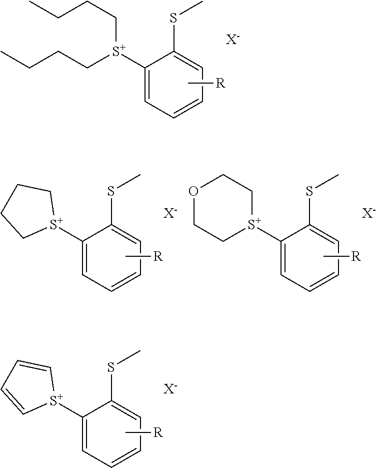

Examples of the above sulfonium salt more preferably include the following compounds. In the following compounds, X⁻ is the same as X⁻ in the above general formula (1).

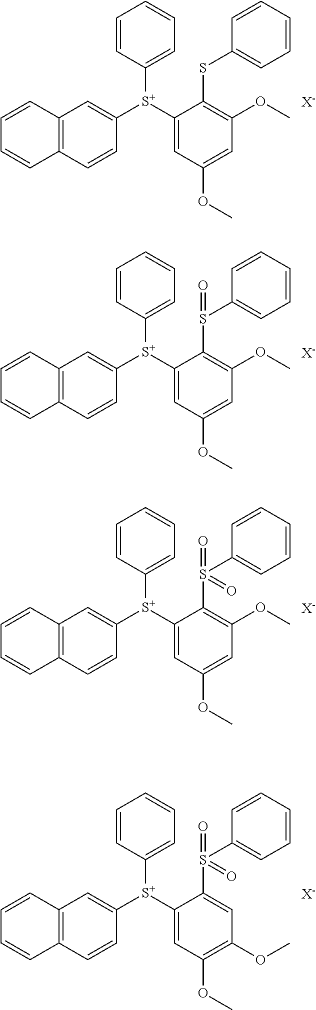

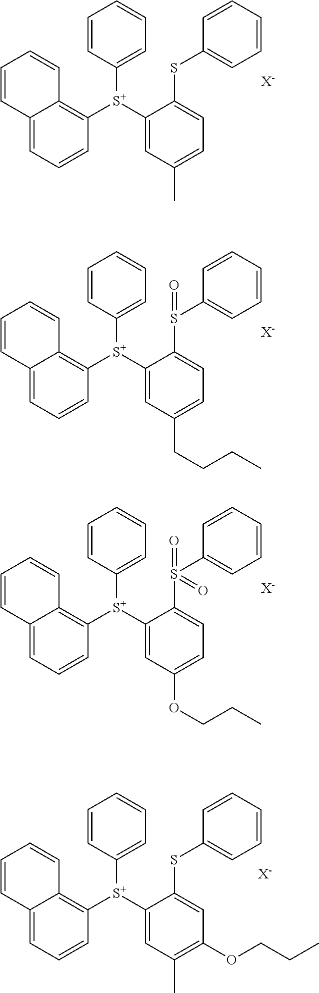

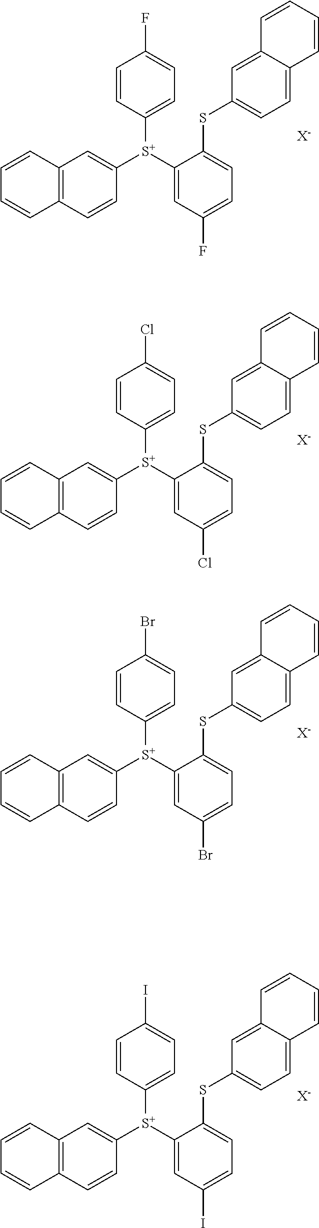

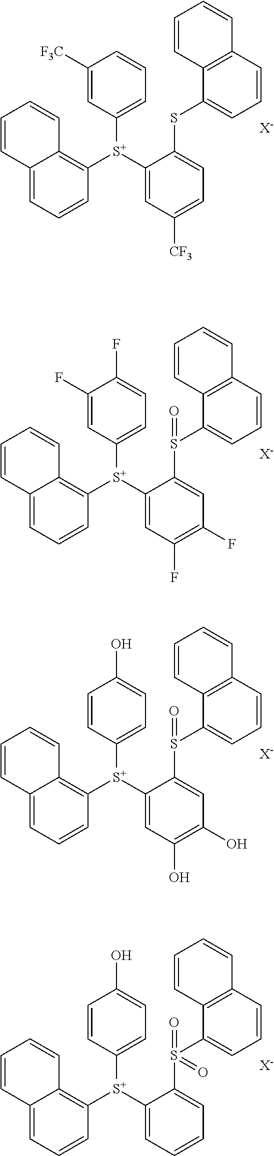

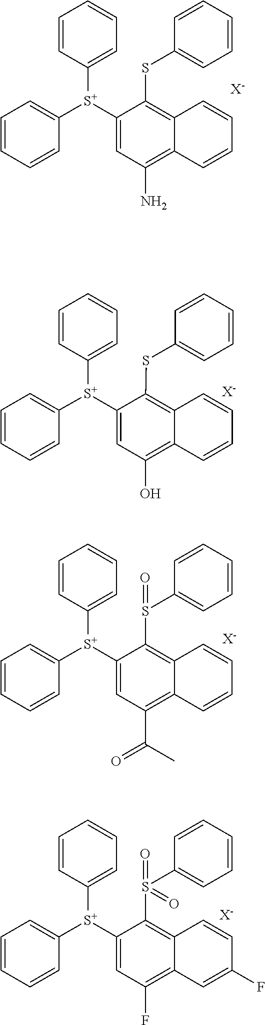

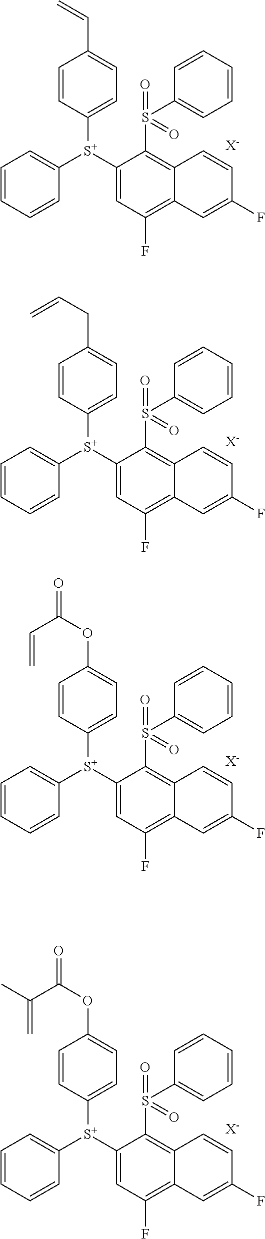

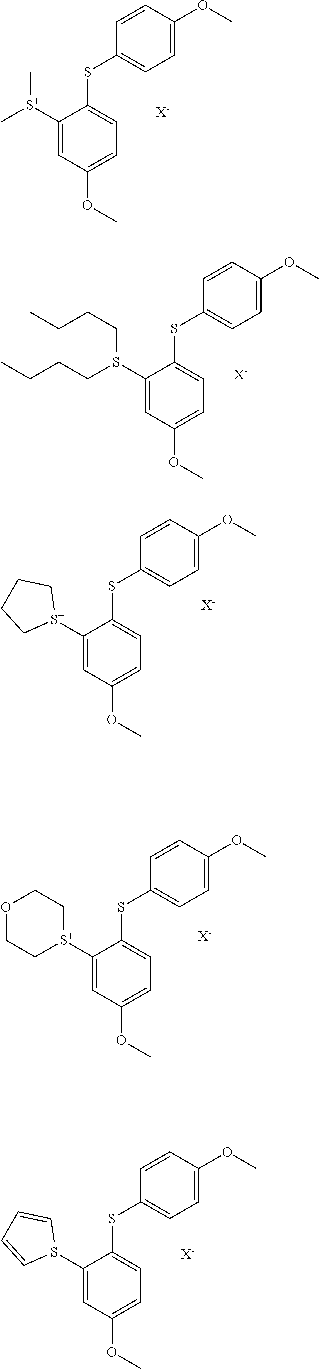

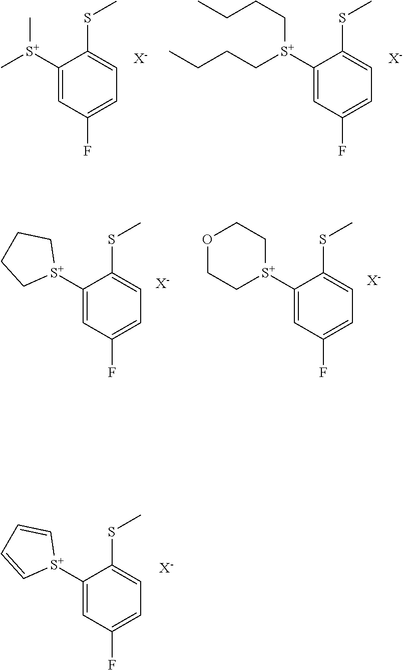

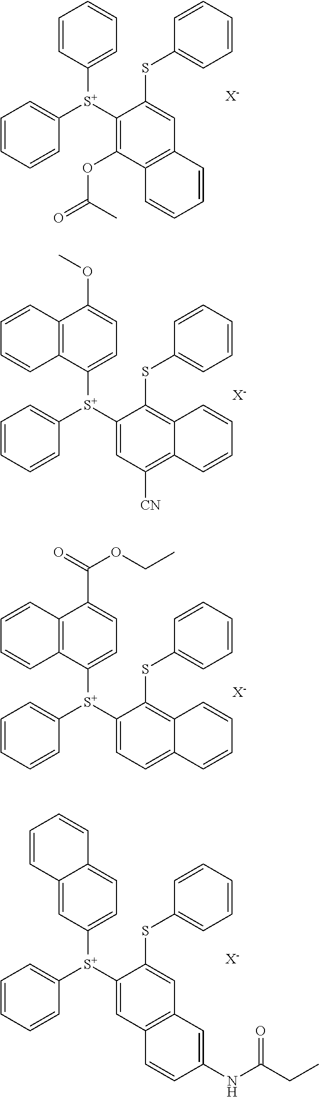

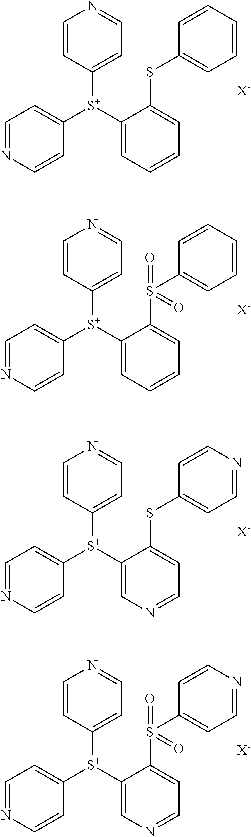

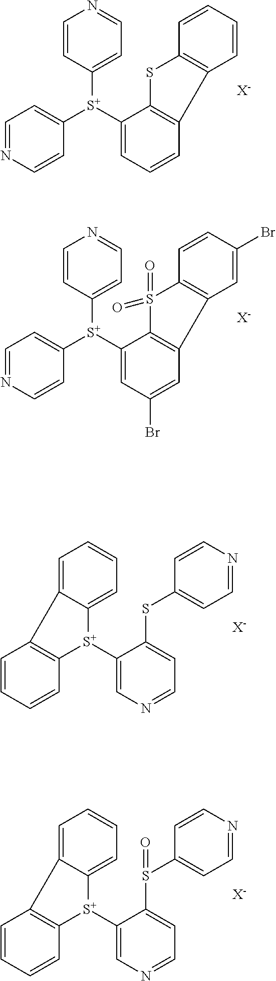

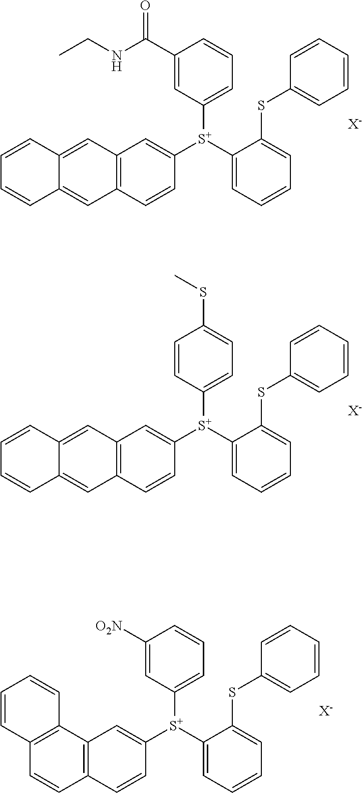

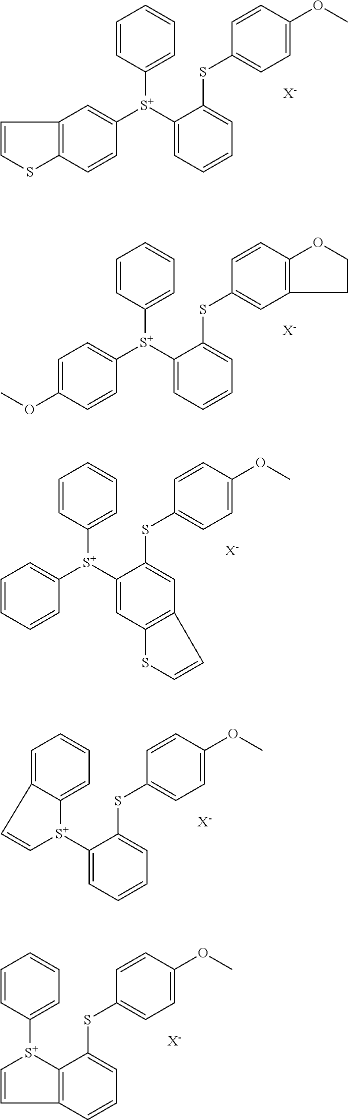

Examples of the above sulfonium salt even more preferably include the sulfonium salts represented by the following general formula (2).

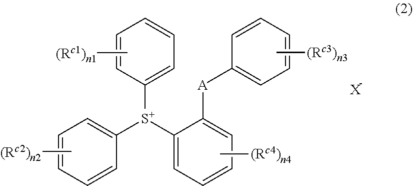

In the above general formula (2), each of A and X⁻ is the same as each of A and X⁻ in the above general formula (1).

Each of R^{c1} to R^{c4} is independently at least any of a monovalent group selected from a group consisting of a monovalent organic group having 1 to 20 carbon atoms, an amino group, a hydroxyl group, a cyano group, a nitro group, and a halogen atom. When there are a plurality of the monovalent organic groups, each of the monovalent organic group is independently selected from the same groups as the monovalent groups represented by the above general formula (3). Examples of the halogen atom include a fluorine atom, a chlorine atom, a bromine atom and an iodine atom.

Examples of R^{c1} to R^{c4} include the same groups as the above substituent (R).

At least two selected from a group consisting of benzene rings bonded to the sulfonio group (S⁺); benzene rings bonded to A; R^{c1}; R^{c2}; R^{c3}; and R^{c4}; optionally form a ring together with the sulfur atom boded to the benzene ring and/or A bonded to the benzene ring, directly with a single bond or via any divalent group selected from a group consisting of an oxygen atom, a sulfur atom, a nitrogen atom-containing group, and an alkylene group.

Each of the nitrogen atom-containing group and the alkylene group is the same as each of the above nitrogen atom-containing group and the above alkylene group in the above general formula (1).

Each of n1 to n3 is integer of 0 to 5. n⁴ is an integer of 0 to 4. n1 to n4 satisfy n1+n2+n3+n4 >0.

Examples of R^{c1} to R^{c4} preferably include: a cyano group, a nitro group, an acyl group, a halogen atom, an halogenated alkyl group and a halogenated aryl group that are substituted at the ortho-position or the para-position; and an alkoxy group, an aryloxy group, a cyano group, a nitro group, an acyl group, a halogen atom, a hydroxyl group, an halogenated alkyl group and a halogenated aryl group that are substituted at the meta-position.

From the viewpoint of sensitivity to an active energy beam, each of R^{c1} to R^{c4} is more preferably a halogen atom and a halogenated alkyl that are substituted at the ortho-position or the para-position, and an alkoxy group, a halogen atom and a halogenated alkyl that are substituted at the meta-position. Halogen atoms such as a fluorine atom and halogenated alkyl groups such as a trifluoromethyl group are particularly preferable.

Each of the ortho-position, meta-position and para-position in the substituent represents the substitution position with respect to the sulfonio group (S⁺) in R^{c1}, R^{c2} and R^{c4}, and the substitution position with respect to A in R^{c3}.

Examples of the above sulfonium salt represented by the above general formula (2) can be exemplified by the following general formulas. In the following general formulas, X⁻ is the same as X⁻ in the above general formula (2).

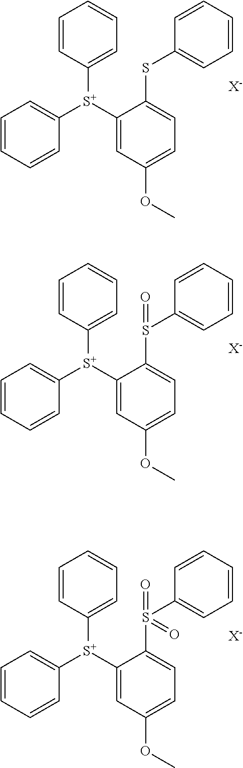

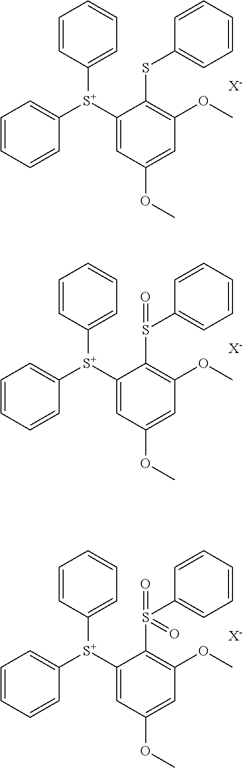

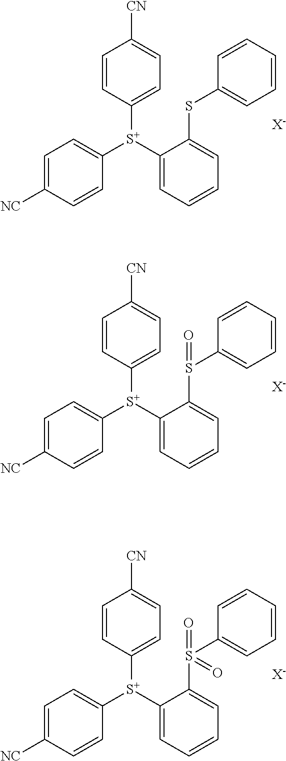

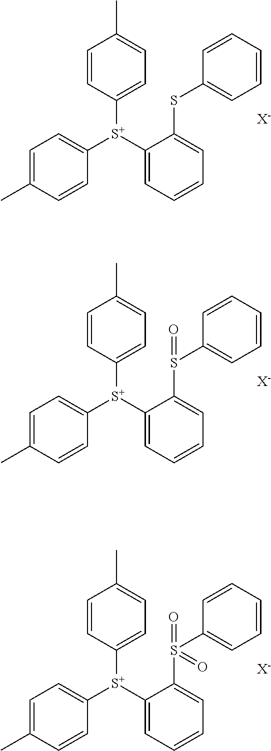

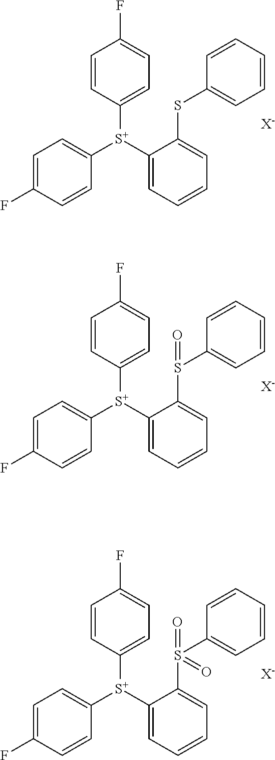

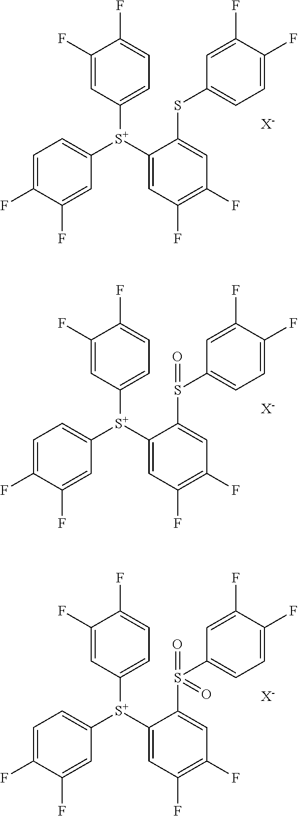

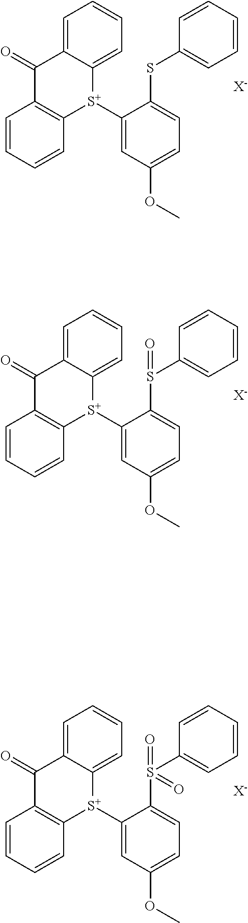

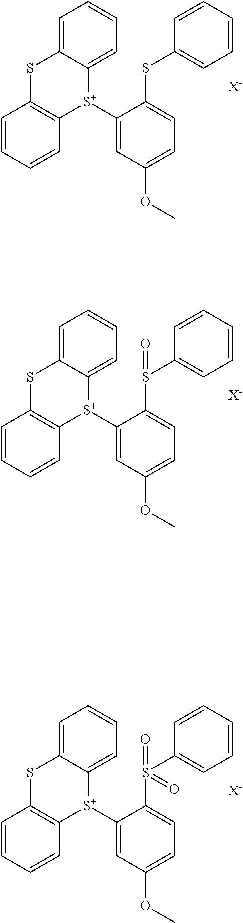

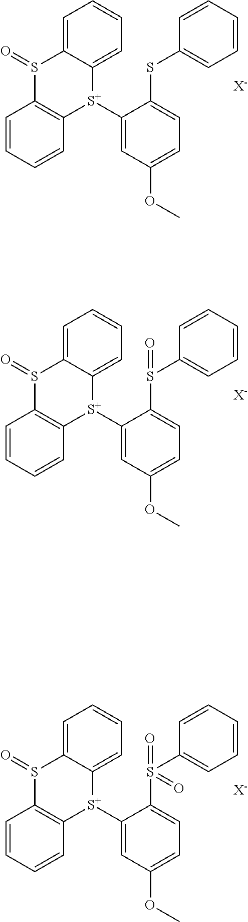

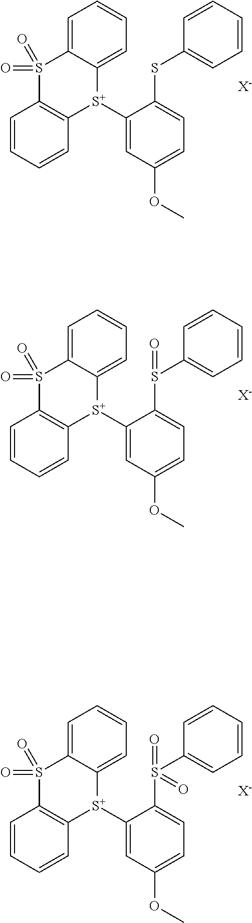

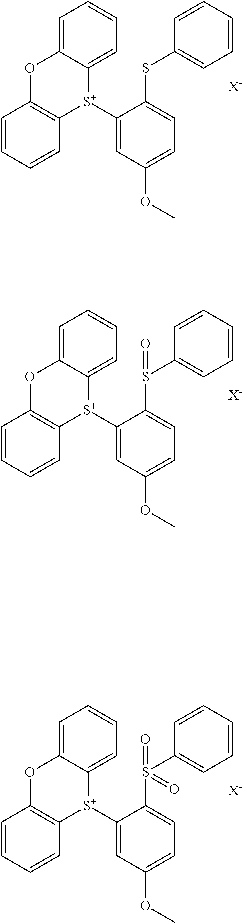

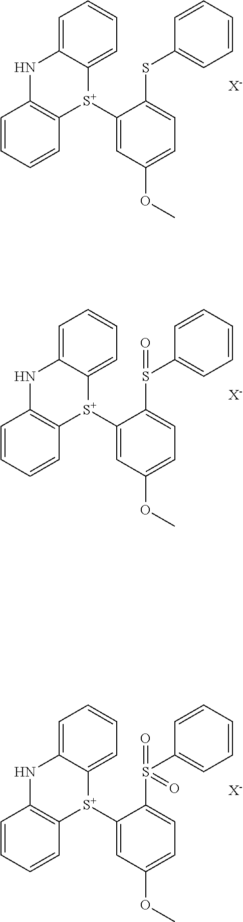

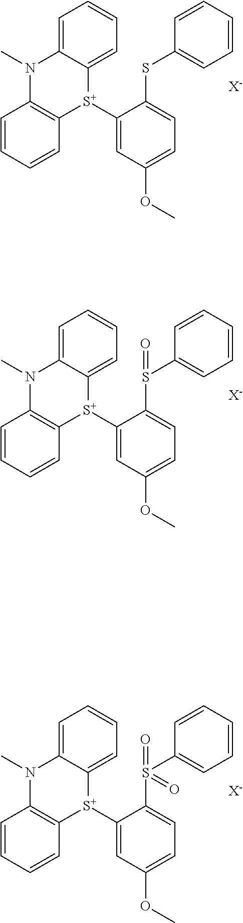

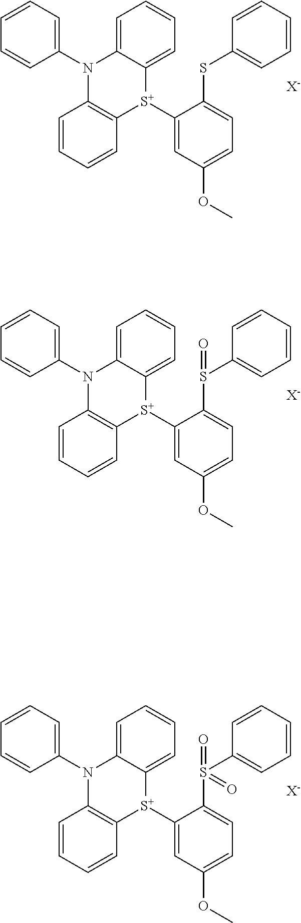

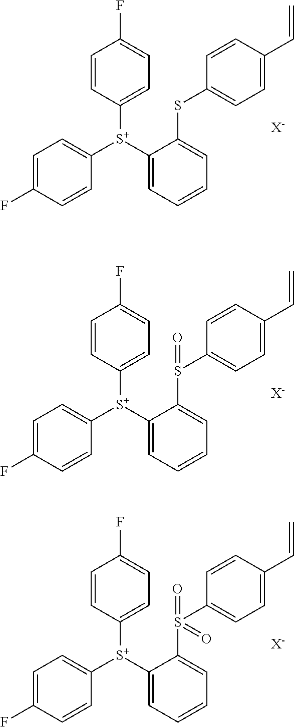

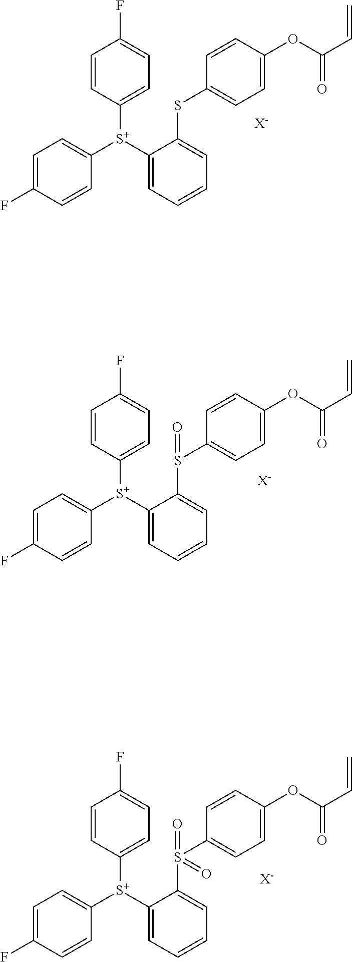

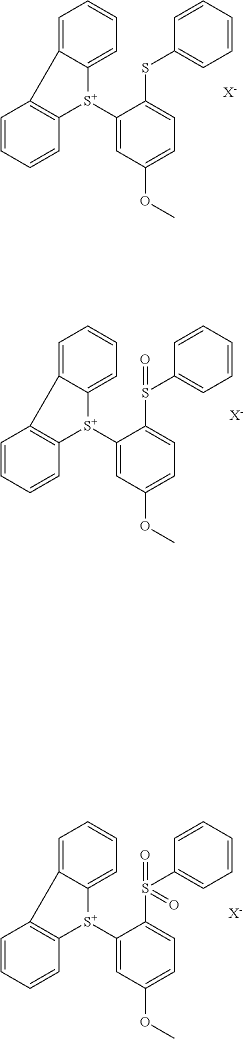

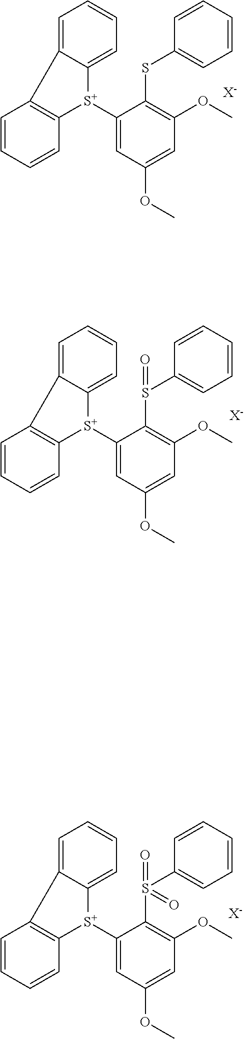

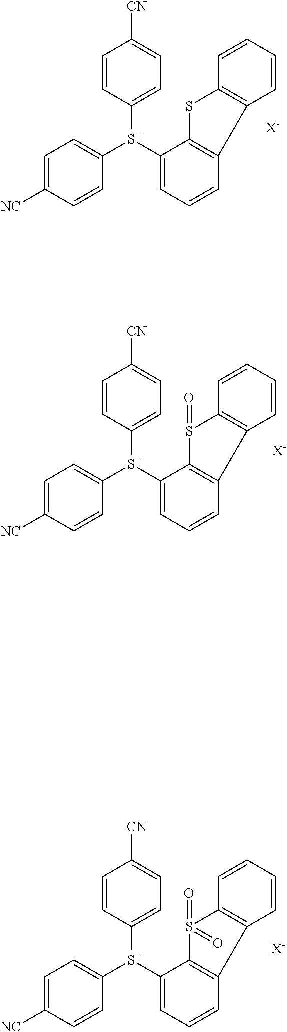

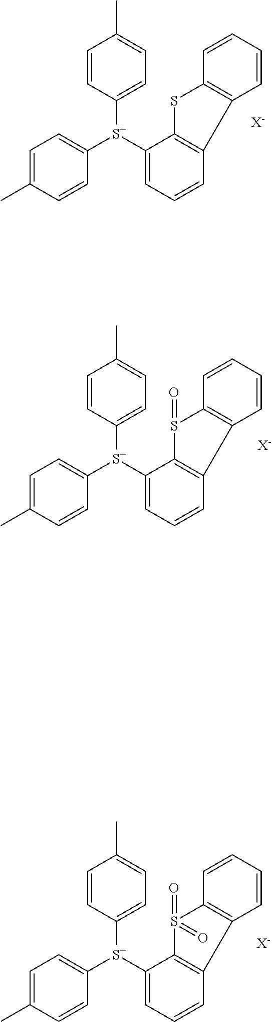

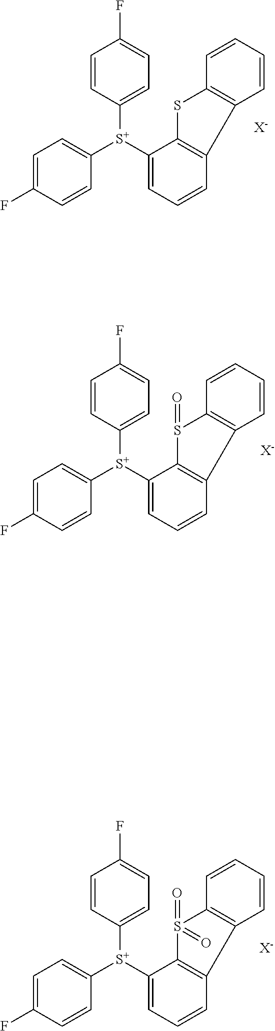

In the above general formulas (1) and (2), X⁻ is an anion.

The anion of the sulfonium salt represented by the above general formulas (1) and (2) indicates a monovalent anion, but the sulfonium salt of one aspect of the present invention may be a divalent or higher anion such as X²⁻ and X³⁻. When the anion is a divalent or higher anion such as X²⁻ and X⁻, the cation corresponds to the anion. Specifically, the substituent in R¹ to R³ and R^{c1} to R^{c4} in the above general formulas (1) and (2) may contain a sulfonio group (S⁺).

Examples of the above anion include a sulfonate anion, a carboxylate anion, an imide anion and a methide anion.

Examples of the above sulfonate anion include anions represented by the following general formula (4).

R¹¹—SO₃⁻ (4)

In the above general formula (4), R¹¹ is a monovalent group represented by the following general formula (5).

R^{a}—L—R^{F}—* (5)

In the above general formula (5), * represents the bond to SO₃⁻ in the above general formula (4). R^{a} is an alkyl group having 1 to 50 carbon atoms which may have a substituent or an aryl group having 6 to 50 carbon atoms which may have a substituent. At least one methylene group contained in the alkyl group may be substituted with at least any divalent heteroatom-containing group selected from the group consisting of —O—, —CO—, —NH—, —S—, —SO—, and —SO₂—. L is at least any divalent group selected from the group consisting of —O—, —CO—, —COO—, —OCO—, —O—CO—O—, —NH—, —NHCO—, —CONH—, —NH—COO—, —OCONH—, —S—, —SO—, and —SO₂—, or a single bond. R^{F} is an alkylene group having 1 to 10 carbon atoms which may have a halogen atom, or a single bond.

Examples of the above alkyl group of R^{a} include the same groups as the above alkyl group in the above substituent (R). The upper limit of the number of carbon atoms of the alkyl group is preferably 30, and more preferably 20.

Examples of the above aryl group of R^{a} include the same groups as the above aryl group in the above substituent (R). The upper limit of the number of carbon atoms of the aryl group is preferably 30, and more preferably 20.

Examples of the substituent that R^{a} may have include the same groups as the above substituent (R).

The number of carbon atoms of R^{a} is a number of carbon atoms including a number of carbon atoms of the above substituent.

Examples of the above alkylene group of R^{F} include: a linear alkylene groups such as a methylene group, an ethylene group and an n-propylene group; branched alkylene groups such as an isopropylene group, an isobutylene group and a tert-butylene group; cyclic alkylene groups such as a cyclopropylene group, a cyclobutylene group, a cyclopentylene group, an adamantandiyl group and an isobornyldiyl group; and combinations thereof.

Examples of the halogen atom that R^{F} may have include a fluorine atom, a chlorine atom, a bromine atom and an iodine atom.

R^{F} having the halogen atom include —CHF—, —CF₂—, —CH (CF₃)—, —CF (CF₃)—, —C (CF₂)₂—, —CHFCF₂—, —CH (CF₃) CF₂—, and combinations thereof.

Examples of the above sulfonate anion include anions having a radically polymerizable group such as a vinyl group, an allyl group, an isopropenyl group, an acryloxy group and a methacryloxy group. When R^{a} is the above radically polymerizable group, the acid generator according to one aspect of the present invention can be used as a polymer component. When the acid generator is used as a polymer component, the diffusion of the generated acid is suppressed, and the resolution and LWR tend to be improved.

When the acid generator according to one aspect of the present invention is used as a polymer component, the number of carbon atoms of R^{a} in the above sulfonate anion represents a number of carbon atoms of the linking group from L to the main chain of the polymer. That is, the number of carbon atoms of R^{a} does not include a number of carbon atoms of the structural unit other than the above sulfonate anion and a number of carbon atoms of the main chain of the polymer.

The following anions can be exemplified as examples of the sulfonate anion represented by the above general formula (4).

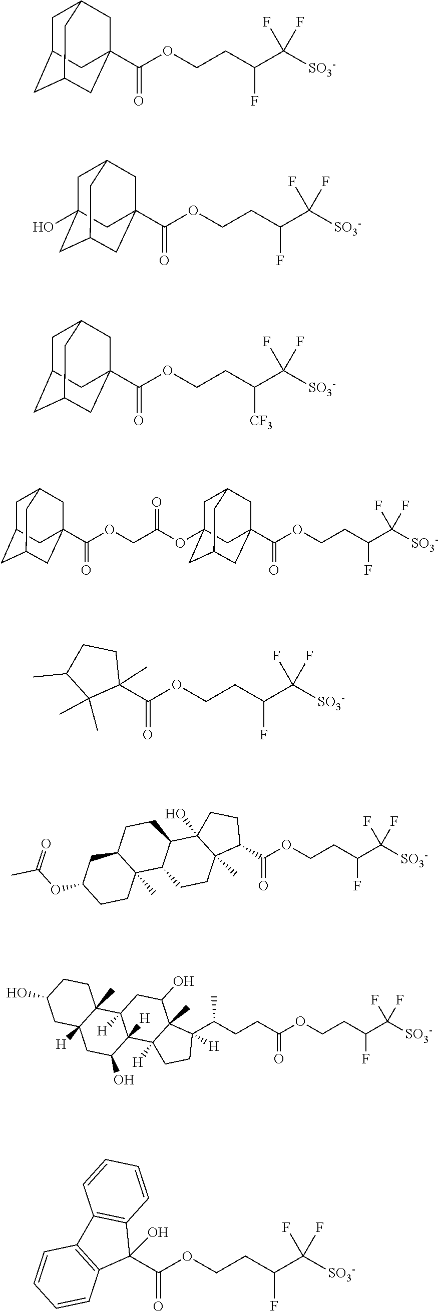

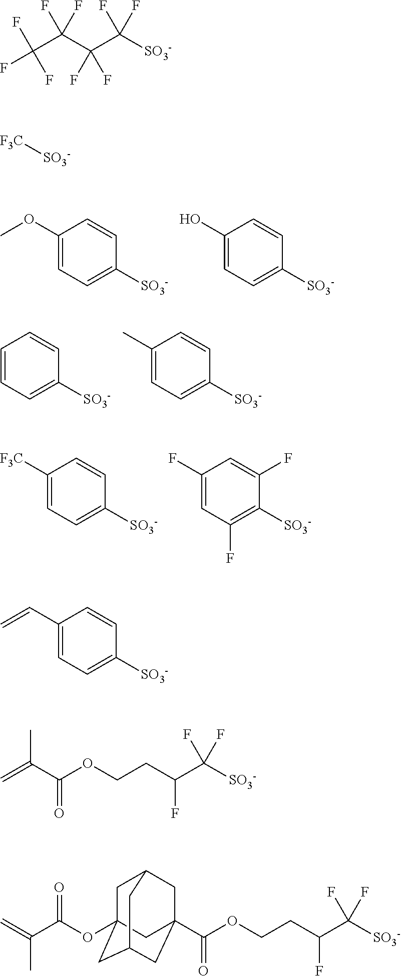

Examples of the above carboxylate anion, the above imide anion and the above methide anion include anions represented by the following general formulas (6) to (8), respectively.

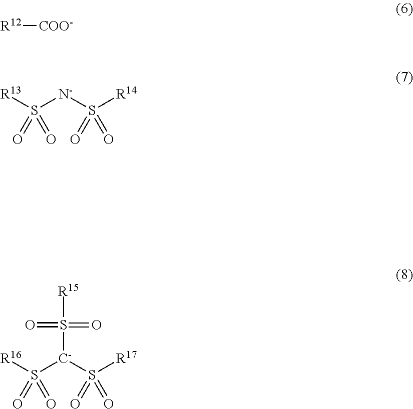

In the above general formulas (6) to (8), each of R¹² to R¹⁷ is independently the same group as the monovalent group represented by the above general formula (5). Each of * in the above general formula (5) in R¹², * in the above general formula (5) in R¹³ and R¹⁴, and * in the above general formula (5) in R¹⁵ to R¹⁷ represents a bond to COO⁻ in the above general formula (6), SO₂N⁻ in the above general formula (7), and SO₂C⁻ of the above general formula (8), respectively.

Two of R¹³ and R¹⁴, and R¹⁵ to R¹⁷ may be bonded each other to form a ring.

### <2> Acid Generator

One aspect of the present invention is an acid generator containing the above sulfonium salt.

In the sulfonium salt represented by the above general formula (1), Ar¹ is substituted with the A at the ortho-position with respect to the sulfonio group (S⁺), and the A is a divalent group selected from the group consisting of —S—, —SO— and —SO₂—. Since the A is located at the ortho-position with respect to the sulfonio group (S⁺) and the sulfonium salt has at least one specific substituent, the above sulfonium salt is efficiently decomposed by irradiation with an active energy beam such as KrF excimer laser, ArF excimer laser, F₂ excimer laser, an electron beam, X-ray and EUV to generate an acid. Therefore, when the above sulfonium salt is used as an acid generator in the resist composition, the resist composition tends to have high sensitivity, and the resist composition tends to be excellent in lithographic properties such as resolution and LWR. Thus, the above sulfonium salt is useful as an acid generator.

### <3> Resist Composition

One aspect of the present invention is a resist composition containing the above acid generator (hereinafter also referred to as the “component (A)”) and an acid reactive compound (hereinafter also referred to as the “component (B)”).

The above resist composition may contain two or more of the above acid generators having different cations and/or anions (hereinafter, also referred to as “component (A1)” and “component (A2)”) together as the component (A). Examples of such a resist composition include a resist composition containing the component (B), the component (A1) that generates an acid that reacts with the component (B), and the component (A2) that functions as a photodegradable base (PDB) with respect to the generated acid from the component (A1).

Further, the component (A) may be a low molecular weight compound, a polymer component, or a mixed component thereof.

When the component (A) is a polymer component, the polymer component may be a homopolymer of the above sulfonium salt, and may be a copolymer whose polymer main chain has the above sulfonium salt as a pendant and a compound other than the sulfonium salt as a pendant, each of which may be included one structural unit. Examples of the structural unit of the above copolymer include: at least one of structural unit having a sulfonium salt as a pendant and, in addition, the component (B) as a pendant; and the structural unit having a phenolic hydroxyl group such as hydroxystyrene and hydroxyvinylnaphthalene; and the like.

When a copolymer containing each of the above sulfonium salt and the component (B) as a structural unit is used, the component (A) and the component (B) may not be further contained as the compounds in addition to the copolymer.

Examples of the component (B) include: a compound having an acid dissociative group (hereinafter, also referred to as “component (B1)”); a compound having a polymerizable group (hereinafter, also referred to as “component (B2)”), which is polymerized by an acid; and a cross-linking agent having a cross-linking function with an acid (hereinafter, also referred to as “component (B3)”) and the like.

The component (B1) is a compound whose solubility in a developer is changed by dissociating the acid dissociative group with an acid to generate a polar group. For example, in the case of a water-based developer using an alkaline developer or the like, it is a compound that is insoluble in the alkaline developer, but becomes soluble in the alkaline developer when an acid is generated from the acid generator by exposure and the acid dissociative group is dissociated in the exposed portion.

In the present invention, the developer may be an alkaline developer, a neutral developer or an organic solvent developer. When the organic solvent developer is used, the compound having an acid dissociative group is a compound whose solubility in an organic solvent developer is lowered when an acid is generated from the above acid generator by exposure and the acid dissociative group is deprotected in the exposed portion.

Specific examples of the above polar group include a carboxyl group, a hydroxyl group, an amino group, a sulfo group (—SO₃H) and the like. Of these, a carboxyl group or a hydroxyl group is preferable. The above acid dissociative group is a group in which the above polar group is protected with a protective group. The protective group is appropriately selected from those usually used as acid dissociative groups in the field of chemically amplified resists, and the examples preferably include a tertiary alkyl ester group, an acetal group, a tetrahydropyranyl group, a siloxy group and a benzyloxy group.

The compound having an acid dissociative group may be a low molecular weight compound, a polymer component, or a mixed component thereof. In the above resist composition, the low molecular weight compound has a polystyrene-equivalent weight average molecular weight of less than 2000, and the polymer component has a polystyrene-equivalent weight average molecular weight of 2000 or more.

As the component (B1), a compound having a hydroxystyrene skeleton or a methacrylate or acrylate skeleton that has the above acid dissociative group as a pendant is preferably used. When the component (B1) is a polymer component, that is, an acid dissociative polymer, the acid dissociative polymer may be used as the base polymer in the resist composition.

When the component (B1) is the above acid dissociative polymer, the acid dissociative polymer has a structural unit containing an acid dissociative group. The acid dissociative polymer further preferably contains a structural unit other than the structural unit containing an acid dissociative group. The structural unit other than the structural unit containing an acid dissociative group is appropriately selected from the structural units usually used in the field of chemically amplified resists. The examples include: a structural unit having at least any of skeletons selected from the group consisting of a lactone skeleton, a sultone skeleton, a lactam skeleton, and the like; and a structural unit having at least any group selected from the group consisting of an ether group, an ester group, a hydroxyl group, a glycidyl group, an oxetanyl group and the like.

Examples of the component (B1) preferably include compounds shown below. The blending ratio of each structural unit and the structure of each structural unit are not limited to the following, and appropriately adjusted depending on the application of the resist composition and the like.

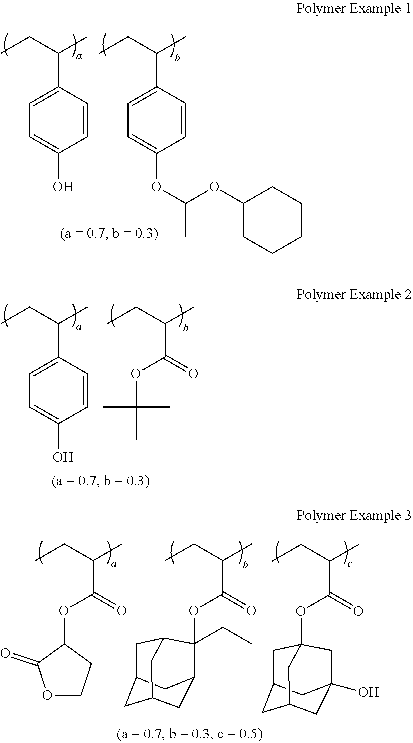

The above component (B2) is a compound whose solubility in a developer changes when the polymerizable group is polymerized with an acid. For example, in the case of a water-based developer, the compound is soluble in the water-based developer, but the solubility in the water-based developer is lowered when an acid is generated from the above acid generator by exposure and the polymerizable group is polymerized in the exposed portion. Also in this case, an organic solvent developer may be used instead of the water-based developer.

Examples of the polymerizable group polymerized by an acid include an epoxy group, an acetal group, an oxetanyl group and the like. As the component (B2), a compound having a styrene skeleton, a methacrylate or acrylate skeleton or the like, each of which has the polymerizable group is preferably used.

The component (B2) may be a polymerizable low molecular weight compound or a polymerizable polymer component. When the component (B2) is a polymerizable polymer component, the polymerizable polymer component may be used as the base polymer in the resist composition.

The above component (B3) is a compound whose solubility in a developer is changed by cross-linking with an acid. For example, in the case of a water-based developer, it is a compound that acts on a compound soluble in the water-based developer and reduces the solubility of the compound in the water-based developer after cross-linking. Specific examples of the component (B3) include a cross-linking agent having an epoxy group, an acetal group, an oxetanyl group and the like. Here, examples of the compound to be cross-linked include a compound having a phenolic hydroxyl group and the like.

The component (B3) may be a crosslinkable low molecular weight compound or a crosslinkable polymer component. When the component (B3) is a crosslinkable polymer component, the crosslinkable polymer component may be used as the base polymer in the resist composition.

More specific examples of the resist composition according to one aspect of the present invention include: a resist composition containing the component (A) and the component (B1); a resist composition containing the component (A) and the component (B2); and a resist composition containing the component (A), the component (B3), and the compound that reacts with the cross-linking agent to change its solubility in a developer; and the like.

The content of the component (A) in the resist composition according to one aspect of the present invention is preferably 1 to 50 parts by mass, more preferably 3 to 30 parts by mass, and even more preferably 5 to 15 parts by mass with respect to 100 parts by mass of the component (B). When the component (A) is contained in the resist composition within the above range, the light transmittance tends to be high even when, for example, the resist composition is used as a permanent film such as an insulating film such as a display body.

When the above acid generator is used as a PDB, the content of the acid generator is preferably 2 to 50 parts by mass, more preferably 3 to 30 parts by mass, and even more preferably 5 to 20 parts by mass with respect to 100 parts by mass of the acid generator that generates an acid that reacts with the component (B).

The resist composition according to one aspect of the present invention may further contain an optional component in addition to the component (A) and the component (B), if necessary.

Specifically, examples of the optional component include a water-repellent polymer (hereinafter, also referred to as “component (C)”) usually used in resist compositions, a polymer other than the component (B) and the component (C) (hereinafter, “component (D)”), an organic solvent (hereinafter, also referred to as “component (E)”), an additive (hereinafter, also referred to as “(F) component”), and an acid generator other than the acid generator according to one aspect of the present invention.

Examples of the above component (C) include a fluorine-containing water-repellent polymer and a silicon-containing water-repellent polymer usually used in an immersion exposure process. The fluorine atom content (mass %) or silicon atom content (mass %) of the component (C) is preferably larger than that of the above component (B) and the above component (D). By adjusting so, when the resist film is formed using the resist composition, the surface free energy of the above water-repellent polymer is relatively low, so that the above water-repellent polymer can be unevenly distributed on the surface of the resist film. This effect suppresses the occurrence of defects by preventing the followability of immersion liquid and liquid residue on the resist film surface and can reduce the amount of elution of the resist component into the immersion liquid, thereby preventing lens contamination.

When the component (C) is used, the blending amount of the component (C) in the resist composition is preferably 0.1 to 30 parts by mass, more preferably 0.5 to 20 parts by mass, and further preferably 1 to 10 parts by mass with respect to 100 parts by mass of the component (B).

The resist composition according to one aspect of the present invention may further contain the component (D) in order to adjust the solubility in the above developer or the adhesion to the substrate. As the component (D), a polymer usually used in the field of chemically amplified resists is appropriately selected. Examples of the component (D) include a polymer having at least any skeleton selected from the group consisting of a lactone skeleton, a sultone skeleton, a lactam skeleton and the like; and a polymer having at least any group selected from the group consisting of an ether group, an ester group, a hydroxyl group, a carboxyl group and the like. The component (D) is a polymer that does not contain the above acid dissociative group-containing structural unit, a fluorine atom, nor a silicon atom.

Examples of the component (D) having a hydroxyl group include: a polymer having a structural unit having a phenolic hydroxyl group such as hydroxystyrene and hydroxyvinylnaphthalene; and a polymer having a structural unit having an alcoholic hydroxyl group such as hydroxyethyl (meth)acrylate and hydroxyadamantyl (meth)acrylate and the like.

Examples of the component (D) having a carboxyl group include: a polymer having an aromatic carboxylic acid structural unit such as vinylbenzoic acid and carboxyphenyl (meth)acrylate; and a polymer having an aliphatic carboxylic acid structural unit such as (meth)acrylic acid, fumaric acid, and maleic acid.

When the component (D) is used, the blending amount of the component (D) in the resist composition is preferably 10 to 150 parts by mass, more preferably 20 to 120 parts by mass, and even more preferably 30 to 100 parts by mass with respect to 100 parts by mass of the component (B).

The above component (E) is appropriately selected from organic solvents usually used in resist compositions. Examples of the component (E) preferably include ethylene glycol monoethyl ether acetate, cyclohexanone, 2-heptanone, propylene glycol monomethyl ether (PGME), propylene glycol monomethyl ether acetate (PGMEA), propylene glycol monomethyl ether propionate, propylene glycol monoethyl ether acetate, methyl 3-methoxypropionate, ethyl 3-ethoxypropionate, methyl β-methoxyisobutyrate, ethyl butyrate, propyl butyrate, methyl isobutyl ketone, ethyl acetate, isoamyl acetate, ethyl lactate, toluene, xylene, cyclohexyl acetate, diacetone alcohol, N-methylpyrrolidone, N,N-dimethylformamide, γ-butyrolactone, N,N-dimethylacetamide, propylene carbonate, ethylene carbonate and the like. These organic solvents may be used alone, or two or more of these may be used in combination.

The above component (F) is appropriately selected from additives usually used in resist compositions. Examples of the component (F) preferably include a quencher, an acidic compound, a dissolution inhibitor, a surfactant, a sensitizer, a stabilizer, a dye, and an EUV absorber such as a metal complex and metal nanoparticles.

### <4> Method for Synthesizing Sulfonium Salt

The sulfonium salt according to one aspect of the present invention can be synthesized by a route represented by the following reaction formula (1), but it can also be synthesized by a route other than the following route depending on the structure of the above sulfonium salt.

First, a condensation reaction is carried out in which a sulfoxide derivative (9) and a sulfide derivative (10) are treated with an acid such as methanesulfonic acid in the presence of a dehydrating agent such as diphosphorus pentoxide to obtain a corresponding sulfonium salt intermediate (11). Subsequently, the anion of the sulfonium salt intermediate (11) is converted to a sulfonium salt (12) by a salt exchange reaction based on a conventional method.

The sulfonium salt (12) corresponds to a sulfonium salt in which the A is —S— in the above general formula (1).

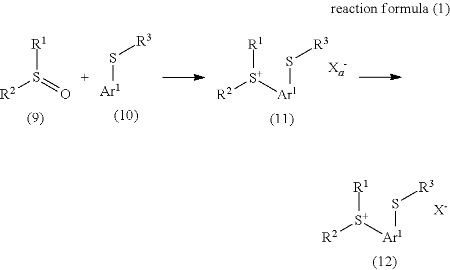

In the above reaction formula (1), each of R¹ to R³, Ar¹ and X⁻ is the same as each of R¹ to R³, Ar¹ and X⁻ in the above general formula (1). Xₐ⁻ is an anion derived from the above acid used in the above condensation reaction.

In the above condensation reaction, examples of the above acid include: alkylsulfonic acids such as methanesulfonic acid, trifluoromethanesulfonic acid, ethanesulfonic acid, and propanesulfonic acid; arylsulfonic acids such as benzenesulfonic acid and p-toluenesulfonic acid; and inorganic acids such as sulfuric acid and fluorosulfonic acid; and the like.

The amount of the above acid used is preferably 1 to 50 mol, and more preferably 10 to 20 mol with respect to 1 mol of the above sulfoxide derivative (9).

Examples of the above dehydrating agent in the above condensation reaction include: inorganic acids such as diphosphorus pentoxide and concentrated sulfuric acid; and acid anhydrides such as trifluoroacetic anhydride, trifluoromethanesulfonic anhydride and acetic anhydride; and the like.

The amount of the above dehydrating agent used is preferably 0.1 to 5 mol, and more preferably 0.2 to 3 mol with respect to 1 mol of the above sulfoxide derivative (9).

In the above condensation reaction, an additive may be added in addition to the above acid and the above dehydrating agent. Examples of the additive include silane compounds such as trimethylsilyl chloride and trimethylsilyl triflate. By adding the silane compound, the above condensation reaction can be promoted.

The amount of the above additive used is preferably 0.1 to 5 mol, and more preferably 0.2 to 3 mol with respect to 1 mol of the above sulfoxide derivative (9).

In the above condensation reaction, a solvent may be added in addition to the above acid, the above dehydrating agent and the above additive.

Examples of the solvent preferably include: aprotic polar solvents such as acetone, acetonitrile and N,N-dimethylformamide; non-polar solvents such as n-pentane, n-hexane and cyclohexane; and halogen-based solvents such as methylene chloride, 1,2-dichloroethane and carbon tetrachloride.

The reaction temperature of the above condensation reaction is preferably −20° C. to 100° C., and more preferably 0° C. to 50° C.

If necessary, the above sulfonium salt (12) can be converted into a sulfonium salt (13) by an oxidation reaction treated with an oxidizing agent such as hydrogen peroxide as shown in the following reaction formula (2). The sulfonium salt (13) corresponds to a sulfonium salt in which the A is —SO— or —SO₂— in the above general formula (1).

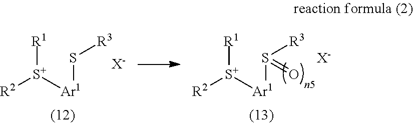

In the above reaction formula (2), each of R¹ to R³, Ar¹ and X⁻ is the same as each of R¹ to R³, Ar¹ and X⁻ in the above general formula (1). n5 is an integer of 1 or 2.

In the above oxidation reaction, examples of the above oxidizing agent include: hydrogen peroxide; percarboxylic acids such as performic acid, peracetic acid, trifluoroperacetic acid, perbenzoic acid and m-chloroperbenzoic acid; and persulfates such as sodium persulfate and potassium persulfate. The percarboxylic acid may be one available as a reagent or may be generated in the system from the corresponding carboxylic acid.

When the sulfoxide compound (n5 is 1) is obtained as the above sulfonium salt (13), the amount of the above oxidizing agent used is preferably 0.8 to 10 mol, and more preferably 1 to 3 mol with respect to 1 mol of the above sulfonium salt (12).

When the sulfone compound (n5 is 2) is obtained as the above sulfonium salt (13), the amount of the above oxidizing agent used is preferably 1 to 20 mol, and more preferably 2 to 6 mol with respect to 1 mol of the above sulfonium salt (12).

Examples of the solvent used for the above oxidation reaction preferably include: water; and a mixed solvent of water and an organic solvent. Examples of the organic solvent include: protonic polar solvents such as methanol, ethanol, 1-propanol and 2-propanol; aprotic polar solvents such as acetone, acetonitrile, dioxane, tetrahydrofuran, N,N-dimethylformamide and dimethylsulfoxide; and non-polar solvents such as methylene chloride, diethyl ether, diisopropyl ether, n-hexane, benzene and toluene.

When the compatibility of the organic solvent with water is low, the above oxidation reaction is carried out in a two-layer system.

The reaction temperature of the above oxidation reaction is preferably −20° C. to 100° C., and more preferably 0° C. to 50° C.

In the above reaction formulas (1) and (2), the sulfonium salt (13) is obtained in the order of the condensation reaction, the salt exchange reaction and the oxidation reaction. However, as shown in the following reaction formula (3), the sulfonium salt (13) may be obtained by performing the oxidation reaction after the condensation reaction and then performing the salt exchange reaction.

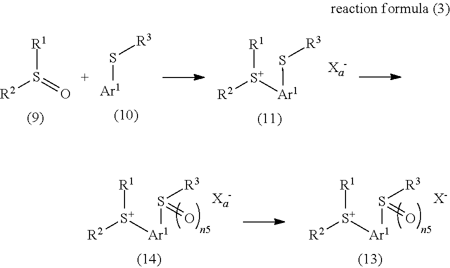

In the above reaction formula (3), each of R¹ to R³, Ar¹, X⁻, Xₐ⁻ and n5 is the same as each of R¹ to R³, Ar¹, X⁻, Xₐ⁻ and n5 in the above reaction formula (1) or (2).

### <5> Method of Manufacturing Device

One aspect of the present invention is a method for manufacturing a device including: a resist film formation step of applying the above resist composition on a substrate to form a resist film; a photolithography step of exposing the resist film to an active energy beam; and a patterning step of developing the exposed resist film to obtain a photoresist pattern.

The above active energy beam is an active energy beam capable of activating the above sulfonium salt to generate an acid, and examples thereof may include visible light, UV, i-line, KrF excimer laser, ArF excimer laser, F₂ excimer laser, EUV (extreme ultraviolet irradiation), X-ray, an electron beam, an ion beam and the like.

For the formation of fine patterns, the above active energy beam is more preferably KrF excimer laser, ArF excimer laser, F₂ excimer laser, EUV, X-ray, an electron beam, an ion beam and the like, and even more preferably EUV and an electron beam.

Except for using the resist composition containing the above acid generator, the usual method for manufacturing a device is followed.

Hereinafter, the present invention will be described based on Examples, but the present invention can be implemented depending on its use and the like.

### <Synthesis of Sulfonium Salt>

(Synthesis Example 1) Synthesis of bis(3,4-dimethoxyphenyl) sulfoxide

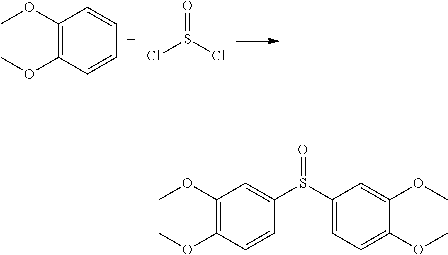

After adding aluminum chloride (24.9 g) to 1,2-dimethoxybenzene (50.0 g), thionyl chloride (11.1 g) is added dropwise to the mixture at 0° C. Then, the temperature of the reaction solution is raised to room temperature and the mixture is stirred for 2 hours. After stirring, the reaction solution is cooled to 0° C., water (150 g) and methylene chloride (150 g) are added dropwise, and the mixture is stirred for 10 minutes. After stirring, the reaction solution is separated and washed twice with 3% by mass sodium hydrogen carbonate aqueous solution (150 g) and three times with water (150 g), and then methylene chloride is distilled off to obtain a crude product. The crude product is purified by silica gel column chromatography (hexane/ethyl acetate=⅓ (volume ratio)) to obtain 21.3 g of bis(3,4-dimethoxyphenyl) sulfoxide.

(Synthesis Example 2) Synthesis of bis(3,4-dimethoxyphenyl) sulfide

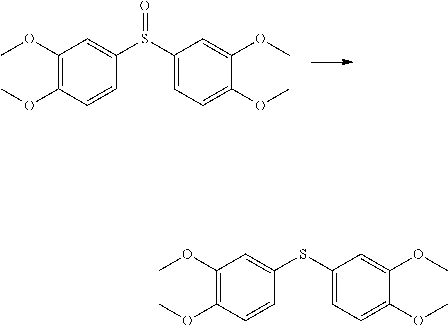

After adding bis(3,4-dimethoxyphenyl) sulfoxide (10.0 g) obtained in Synthesis Example 1 and triphenylphosphine (8.6 g) to tetrahydrofuran (30 g), thionyl chloride (5.1 g) is dropped to the mixture at 0° C. Then, the temperature of the reaction solution is raised to room temperature and the reaction solution is stirred for 1 hour. After stirring, hexane (100 g) is added to the reaction solution, and the precipitate is removed by filtration to recover the reaction solution. The recovered reaction solution is washed three times with water (50 g) and then hexane is distilled off to obtain a crude product. The crude product is purified by silica gel column chromatography (hexane/ethyl acetate=4/1 (volume ratio)) to obtain 7.4 g of bis(3,4-dimethoxyphenyl) sulfide.

### (Synthesis Example 3) Synthesis of Sulfonium Salt 1

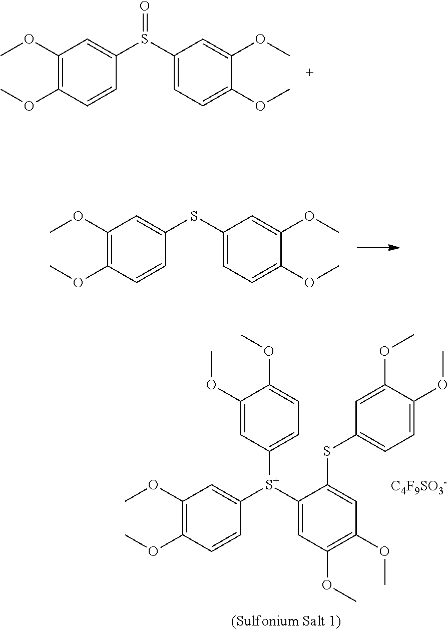

Bis(3,4-dimethoxyphenyl) sulfoxide (5.0 g) obtained in Synthesis Example 1, bis(3,4-dimethoxyphenyl) sulfide (4.8 g) obtained in Synthesis Example 2, and phosphorus pentoxide (5.2 g) are dissolved in methanesulfonic acid (25.0 g) and the mixture is stirred at 40° C. for 3 hours. After stirring, pure water (75 g) is added, and the mixture is further stirred for 10 minutes. Then, potassium nonafluorobutanesulfonate (5.2 g) and methylene chloride (100 g) are added to the mixture and the mixture is further stirred for 1 hour. The reaction solution is separated and washed three times with pure water (75 g), and then methylene chloride is distilled off to obtain a crude product. The crude product is purified by silica gel column chromatography (methylene chloride/methanol=90/10 (volume ratio)) to obtain 9.8 g of Sulfonium Salt 1.

### (Synthesis Example 4) Synthesis of Sulfonium Salt 2

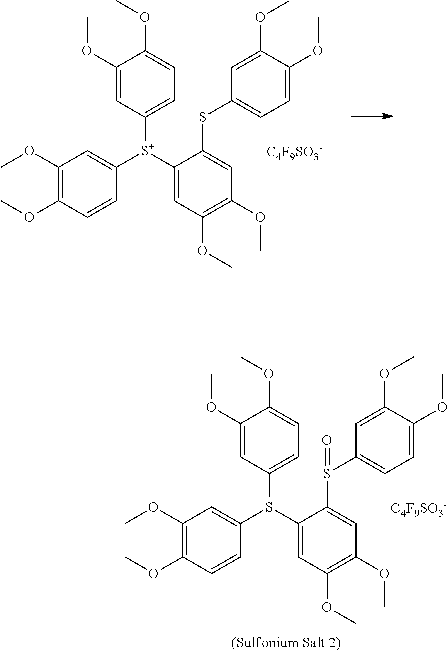

The Sulfonium Salt 1 (4.0 g) obtained in Synthesis Example 3 is added to methanol (8.0 g), and the reaction solution is cooled to 0° C. Then, 35% by mass hydrogen peroxide aqueous solution (0.42 g) is added dropwise, the temperature of the reaction solution is raised to room temperature, and the mixture is stirred for 1 hour. After stirring, methylene chloride (16.0 g) is added to the reaction solution and the mixture is stirred for 10 minutes. Then, the reaction solution is separated, washed three times with water (8 g), and then methylene chloride is distilled off to obtain a crude product. The crude product is purified by silica gel column chromatography (methylene chloride/methanol=90/10 (volume ratio)) to obtain 3.5 g of Sulfonium Salt 2.

### (Synthesis Example 5) Synthesis of Sulfonium Salt 3

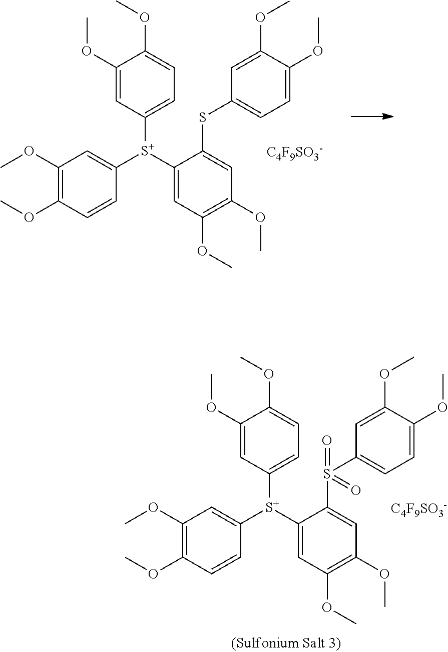

The same operation as in Synthesis Example 4 is carried out to obtain 3.9 g of Sulfonium Salt 3, except that 35% by mass hydrogen peroxide aqueous solution (0.90 g) is used instead of 35% by mass hydrogen peroxide aqueous solution (0.42 g).

(Synthesis Example 6) Synthesis of bis(3,4-difluorophenyl) sulfoxide

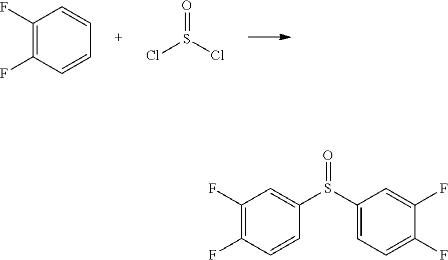

The same operation as in Synthesis Example 1 is carried out to obtain bis(3,4-difluorophenyl) sulfoxide, except that 1,2-difluorobenzene is used instead of 1,2-dimethoxybenzene and the reaction solution is heated to 80° C. for reaction.

(Synthesis Example 7) Synthesis of bis(3,4-difluorophenyl) sulfide

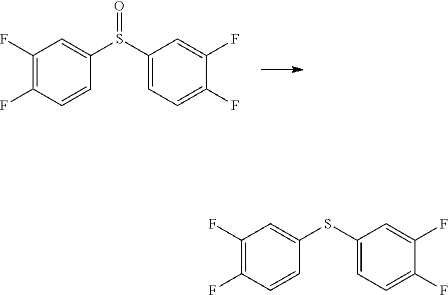 [SMILES: O=S(c1ccc(F)c(F)c1)c1ccc(F)c(F)c1 ; Fc1ccc(Sc2ccc(F)c(F)c2)cc1F]

The same operation as in Synthesis Example 2 is carried out to obtain bis(3,4-difluorophenyl) sulfide, except that bis(3,4-diflulophenyl) sulfoxide obtained in Synthesis Example 6 is used instead of bis(3,4-dimethoxyphenyl) sulfoxide.

### (Synthesis Example 8) Synthesis of Sulfonium Salt 4

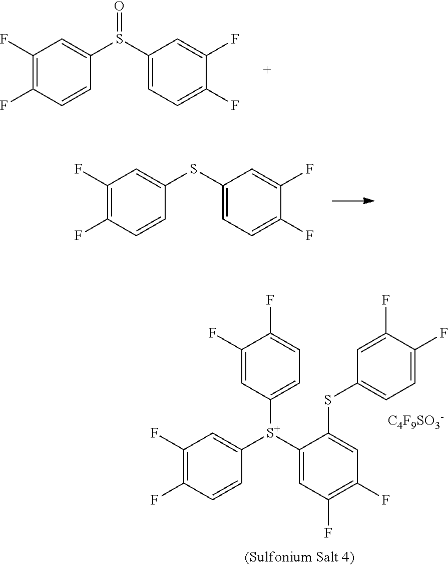

The same operation as in Synthesis Example 3 is carried out to obtain the Sulfonium Salt 4, except that: bis(3,4-difluorophenyl) sulfoxide obtained in Synthesis Example 6 is used instead of bis(3,4-dimethoxyphenyl) sulfoxide; and bis(3,4-difluorophenyl) sulfide obtained in Synthesis Example 7 is used instead of the bis(3,4-dimethoxyphenyl) sulfide.

### (Synthesis Example 9) Synthesis of Sulfonium Salt 5

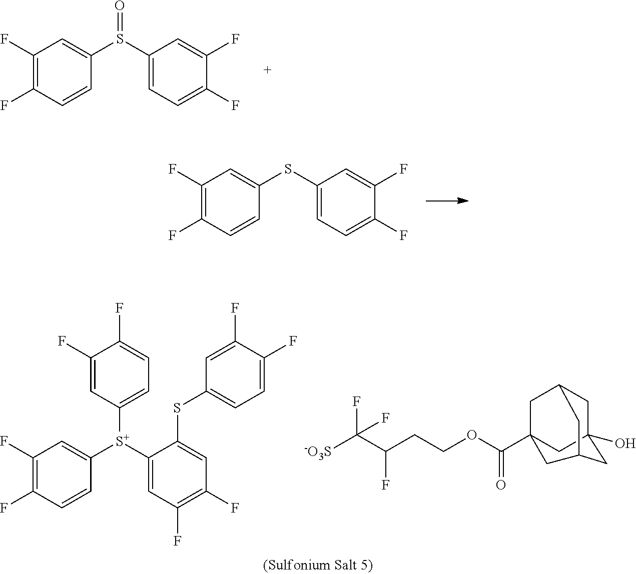

The same operation as in Synthesis Example 8 is carried out to obtain Sulfonium Salt 5, except that sodium 4-(3-hydroxyadamantylcarbonyloxy)-1,1,2-trifluorobutanesulfonate is used instead of potassium nonafluorobutanesulfonate.

(Synthesis Example 10) Synthesis of bis[3,5-bis(trifluoromethyl)phenyl] sulfoxide

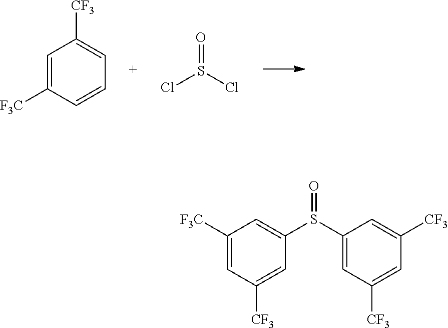

The same operation as in Synthesis Example 1 is carried out to obtain bis[3,5-bis(trifluoromethyl)phenyl]sulfoxide, except that 1,3-bis(trifluoromethyl)benzene is used instead of 1,2-dimethoxybenzene and the reaction solution is heated to 80° C. for reaction.

### (Synthesis Example 11) Synthesis of Sulfonium Salt 6

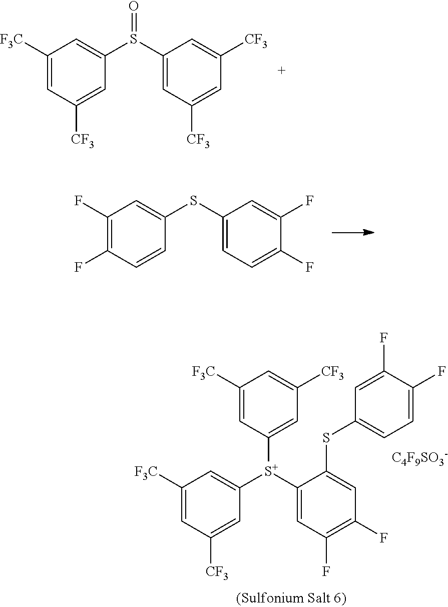

The same operation as in Synthesis Example 8 is carried out to obtain Sulfonium Salt 6, except that the bis[3,5-bis(trifluoromethyl)phenyl] sulfoxide obtained in Synthesis Example 10 is used instead of bis(3,4-difluorophenyl) sulfoxide.

### (Synthesis Example 12) Synthesis of Sulfonium Salt 7

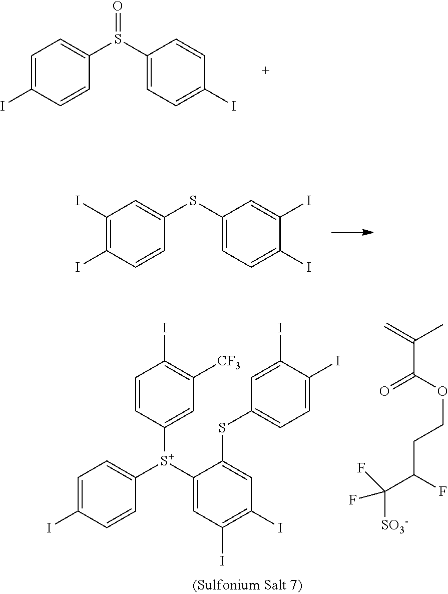

The same operation as in Synthesis Example 3 is carried out to obtain Sulfonium Salt 7, except that: bis(4-iodophenyl) sulfoxide is used instead of bis(3,4-dimethoxyphenyl) sulfoxide; bis(3,4-diiodophenyl) sulfide is used instead of bis(3,4-dimethoxyphenyl) sulfide; and sodium 4-methacryloyloxy-1,1,2-trifluorobutanesulfonate is used instead of potassium nonafluorobutanesulfonate. Bis(4-iodophenyl) sulfoxide and bis(3,4-diiodophenyl) sulfide can be synthesized by using iodobenzene and 1,2-diiodobenzene and following Synthesis Examples 1 and 2.

(Synthesis Example 13) Synthesis of dibenzothiophene-5-oxide

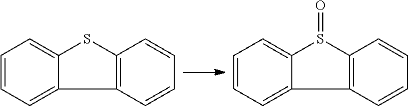 [SMILES: c1ccc2c(c1)sc1ccccc12 ; O=S1c2ccccc2-c2ccccc21]

Dibenzothiophene (15.0 g) is dissolved in formic acid (75.0 g), and 35% by mass hydrogen peroxide aqueous solution (8.7 g) is added dropwise thereto under ice cooling. Then, the temperature is raised to room temperature and the mixture is stirred for 5 hours. After stirring, pure water (200 g) is added dropwise to the reaction solution to precipitate a solid. The precipitated solid is separated by filtration, washed three times with pure water (40 g), and then dried to obtain a crude crystal. The crude crystal is recrystallized by using acetone (100 g) and ethanol (200 g) to obtain 12.1 g of dibenzothiophene-5-oxide.

### (Synthesis Example 14) Synthesis of Sulfonium Salt 8

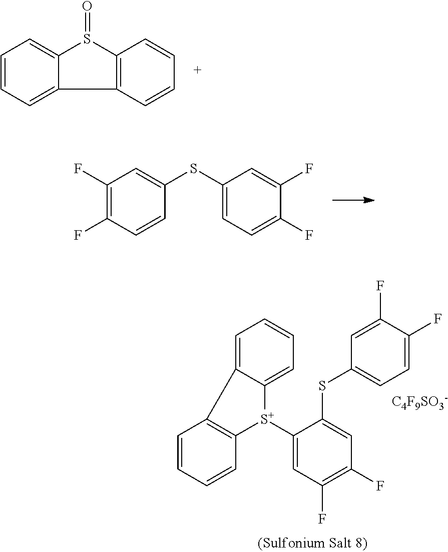

The same operation as in Synthesis Example 8 is carried out to obtain Sulfonium Salt 8, except that the dibenzothiophene-5-oxide obtained in Synthesis Example 13 is used instead of bis(3,4-difluorophenyl) sulfoxide.

### (Synthesis Example 15) Synthesis of Sulfonium Salt 9

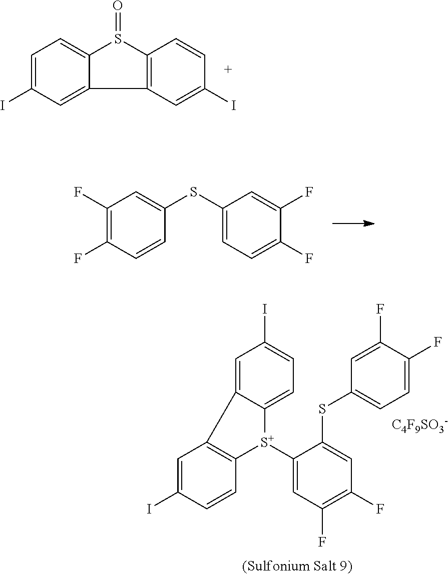

The same operation as in Synthesis Example 14 is carried out to obtain Sulfonium Salt 9, except that 2,8-diiododibenzothiophene-5-oxide is used instead of dibenzothiophene-5-oxide. 2,8-diiododibenzothiophene-5-oxide can be synthesized by using 2,8-diiododibenzothiophene and following Synthesis Example 13.

### (Synthesis Example 16) Synthesis of Sulfonium Salt 10

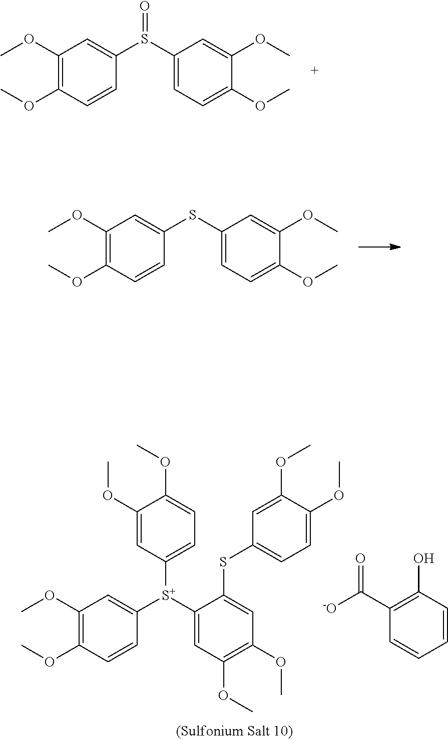

The same operation as in Synthesis Example 3 is carried out to obtain Sulfonium Salt 10, except that sodium salicylate is used instead of potassium nonafluorobutanesulfonate.

### <Synthesis of Polymer>

### (Synthesis Example 17) Synthesis of Polymer A

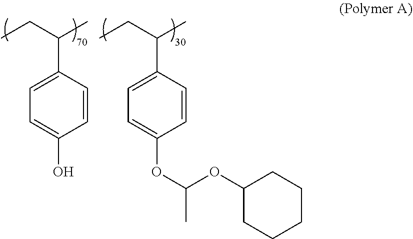

Polyhydroxystyrene (weight average molecular weight 8000) (8.0 g) and 35% by mass hydrochloric acid (0.010 g) are dissolved in dehydrated dioxane (28.0 g) to prepare a polyhydroxystyrene solution. A solution prepared by dissolving cyclohexyl vinyl ether (2.73 g) in dehydrated dioxane (2.80 g) is added dropwise to the above polyhydroxystyrene solution over 30 minutes. After the dropping, the reaction solution is heated to 40° C. and stirred for 2 hours. After stirring and cooling, N,N-dimethylaminopyridine (0.014 g) is added. Then, the polymer is precipitated by dropping the solution into pure water (260 g). The solid obtained by separating the precipitate by vacuum filtration is washed twice with pure water (300 g) and then vacuum dried to obtain 9.2 g of the above Polymer A as a white solid.

### (Synthesis Example 18) Synthesis of Polymer B

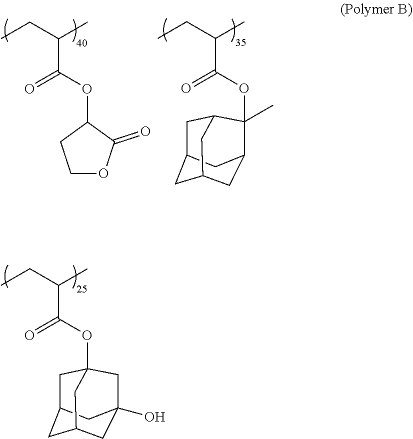

α-Methacryloxy-y-butyrolactone (5.0 g), 2-methyl-2-adamantyl methacrylate (6.0 g), 3-hydroxy-1-adamantyl methacrylate (4.3 g), and dimethyl-2,2′-azobis(2-methylpropionate) (0.51 g) are dissolved in propylene glycol monomethyl ether acetate (PGMEA) (26.0 g), and the mixture is deoxidized. The deoxidized product is added dropwise to PGMEA (7.5 g) preheated to 85° C. over 4 hours. After stirring for 2 hours, the mixture is cooled. After cooling, the mixture is dropped into hexane (180 g) to perform reprecipitation. The reprecipitate is filtered, dispersed in and washed with hexane (70 g), filtered again, and then vacuum dried to obtain 8.5 g of the above Polymer B as a white solid.

### <Preparation and Evaluation of Resist Composition>

### EXAMPLES 1 TO 10

The above Sulfonium Salt 1 (10.2 parts by mass), Polymer A (100 parts by mass), and triethanolamine (0.20 parts by mass) are dissolved in propylene glycol monomethyl ether acetate (PGMEA) (1250 parts by mass). The obtained solution is filtered through a PTFE filter to prepare a resist composition of Example 1 below. Next, the resist composition is rotationally applied on a silicon wafer and then prebaked on a hot plate at 110° C. for 60 seconds to obtain a resist film having a film thickness of 200 nm. An electron beam drawing apparatus is used to draw a line-and-space pattern of 200 nm on this resist film with an electron beam of 30 keV. Then, post-baking is performed at 110° C. for 60 seconds. Then, the post-baked film is developed with a 2.38% by mass of aqueous solution of tetramethylammonium hydroxide for 60 seconds and rinsed with pure water for 30 seconds to obtain a pattern.

The obtained pattern is observed with a microscope, and the irradiation amount of the electron beam when the resist film is completely peeled off is defined as the sensitivity [μC/cm²]. When the sensitivity of Comparative Example 1 below is set to 1.00, the sensitivity of the resist composition of Example 1 is calculated as a relative value.

The resolution and LWR are evaluated as follows. The resolution and LWR are measured using the resist composition of Comparative Example 1 below. When the resolution and LWR values of Comparative Example 1 are set to 1.00, respectively, the resolution and LWR of the resist composition of Example 1 are calculated as relative values.

For each of the Sulfonium Salts 2 to 9, the resist compositions of Examples 2 to 9 are prepared and evaluated in the same manner as described above. The amount of each of the sulfonium salts 2 to 9 added is adjusted to be the same molar amount as that of the Sulfonium Salt 1 of Example 1. Further, in Example 10, a resist composition in which triethanolamine (0.20 parts by mass) in the resist composition of Example 1 is replaced with Sulfonium Salt 10 (1.00 part by mass) is evaluated. The results are shown in Table 1.

### COMPARATIVE EXAMPLES 1 TO 3

A resist composition is prepared and evaluated in the same manner as above except that the Comparative Sulfonium Salts 1 to 3 represented by the following formulas are used instead of the Sulfonium Salt 1.

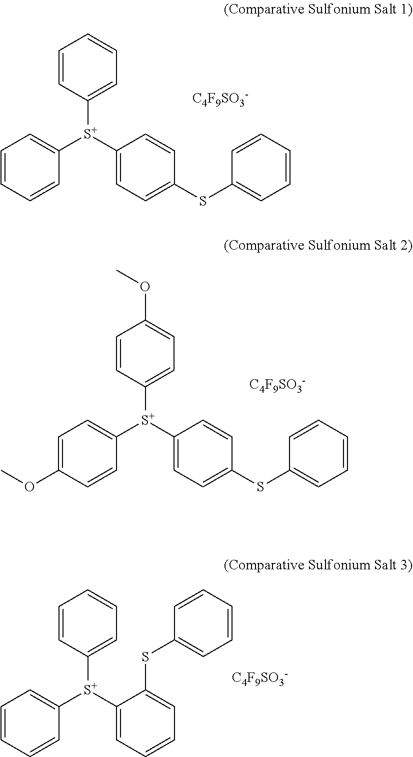

### EXAMPLES 11 TO 20

The resist compositions of Examples 11 to 20 are prepared in the same manner as above except that: the Sulfonium Salt 1 (10.2 parts by mass) is replaced with (6.79 parts by mass); and the Polymer B is used instead of the Polymer A. An ArF excimer laser stepper (wavelength 193 nm) is used instead of the electron beam drawing apparatus, and the exposure evaluation is performed in the same manner as described above. The irradiation amount of the ArF excimer laser is defined as the sensitivity [mJ/cm²]. The resolution and LWR are also evaluated in the same manner as above. The results are shown in Table 2.

### COMPARATIVE EXAMPLES 4 TO 6

A resist composition is prepared and evaluated in the same manner as in Examples 11 to 20 except that the comparative sulfonium salts 1 to 3 are used instead of the Sulfonium Salt 1.

TABLE 1

| | Polymer | | Solvent | | Quencher | | Sulfonium Salt | | Evaluation Result | | |
|---|---|---|---|---|---|---|---|---|---|---|---|
| | | Part by | | Part by | | Part by | | Part by | | | |
| | Type | Mass | Type | Mass | Type | Mass | Type | Mass | Sensitivity | Resolution | LWR |
| Example1 | Polymer A | 100 | PGMEA | 1250 | Triethanolamine | 0.20 | Sulfonium | 10.2 | 0.96 | 0.98 | 0.98 |
| | | | | | | | Salt 1 | | | | |
| Example2 | | | | | | | Sulfonium | 10.4 | 0.94 | 0.98 | 0.98 |
| | | | | | | | Salt 2 | | | | |
| Example3 | | | | | | | Sulfonium | 10.5 | 0.92 | 0.98 | 0.97 |
| | | | | | | | Salt 3 | | | | |
| Example4 | | | | | | | Sulfonium | 9.11 | 0.89 | 0.97 | 0.98 |
| | | | | | | | Salt 4 | | | | |
| Example5 | | | | | | | Sulfonium | 10.1 | 0.89 | 0.95 | 0.95 |
| | | | | | | | Salt 5 | | | | |
| Example6 | | | | | | | Sulfonium | 11.3 | 0.86 | 0.96 | 0.97 |
| | | | | | | | Salt 6 | | | | |
| Example7 | | | | | | | Sulfonium | 15.8 | 0.90 | 0.98 | 0.98 |
| | | | | | | | Salt 7 | | | | |
| Example8 | | | | | | | Sulfonium | 8.28 | 0.90 | 0.96 | 0.97 |
| | | | | | | | Salt 8 | | | | |
| Example9 | | | | | | | Sulfonium | 11.1 | 0.88 | 0.95 | 0.97 |
| | | | | | | | Salt 9 | | | | |
| Example10 | | | | | Sulfonium | 1.00 | Sulfonium | 10.2 | 0.96 | 0.96 | 0.95 |
| | | | | | Salt 10 | | Salt 1 | | | | |
| Comparative | | | | | Triethanolamine | 0.20 | Comparison | 7.50 | 1.00 | 1.00 | 1.00 |
| Example 1 | | | | | | | Sulfonium | | | | |
| | | | | | | | Salt 1 | | | | |
| Comparative | | | | | | | Comparison | 8.18 | 0.97 | 0.98 | 1.03 |
| Example 2 | | | | | | | Sulfonium | | | | |
| | | | | | | | Salt 2 | | | | |
| Comparative | | | | | | | Comparison | 7.50 | 1.02 | 1.00 | 0.98 |
| Example 3 | | | | | | | Sulfonium | | | | |
| | | | | | | | Salt 3 | | | | |

TABLE 2

| | Polymer | | Solvent | | Quencher | | Sulfonium Salt | | Evaluation Result | | |
|---|---|---|---|---|---|---|---|---|---|---|---|
| | | Part by | | Part by | | Part by | | Part by | | | |
| | Type | Mass | Type | Mass | Type | Mass | Type | Mass | Sensitivity | Resolution | LWR |
| Example 11 | Polymer B | 100 | PGMEA | 1250 | Triethanolamine | 0.20 | Sulfonium | 6.79 | 0.94 | 0.97 | 0.98 |
| | | | | | | | Salt 1 | | | | |
| Example 12 | | | | | | | Sulfonium | 6.91 | 0.93 | 0.97 | 0.98 |
| | | | | | | | Salt 2 | | | | |
| Example 13 | | | | | | | Sulfonium | 7.03 | 0.91 | 0.96 | 0.97 |
| | | | | | | | Salt 3 | | | | |
| Example 14 | | | | | | | Sulfonium | 6.07 | 0.95 | 0.97 | 0.98 |
| | | | | | | | Salt 4 | | | | |
| Example 15 | | | | | | | Sulfonium | 6.72 | 0.95 | 0.95 | 0.95 |
| | | | | | | | Salt 5 | | | | |
| Example 16 | | | | | | | Sulfonium | 7.56 | 0.94 | 0.96 | 0.97 |
| | | | | | | | Salt 6 | | | | |
| Example 17 | | | | | | | Sulfonium | 10.5 | 0.95 | 0.98 | 0.98 |
| | | | | | | | Salt 7 | | | | |
| Example 18 | | | | | | | Sulfonium | 5.52 | 0.97 | 0.96 | 0.97 |
| | | | | | | | Salt 8 | | | | |
| Example 19 | | | | | | | Sulfonium | 7.40 | 0.96 | 0.95 | 0.97 |
| | | | | | | | Salt 9 | | | | |
| Example 20 | | | | | Sulfonium | 1.00 | Sulfonium | 6.79 | 0.94 | 0.95 | 0.96 |
| | | | | | Salt 10 | | Salt 1 | | | | |
| Comparative | | | | | Triethanolamine | 0.20 | Comparison | 5.00 | 1.00 | 1.00 | 1.00 |
| Example 4 | | | | | | | Sulfonium | | | | |
| | | | | | | | Salt 1 | | | | |
| Comparative | | | | | | | Comparison | 5.45 | 0.98 | 0.98 | 1.04 |
| Example 5 | | | | | | | Sulfonium | | | | |
| | | | | | | | Salt 2 | | | | |
| Comparative | | | | | | | Comparison | 5.00 | 1.00 | 1.00 | 0.99 |
| Example 6 | | | | | | | Sulfonium | | | | |
| | | | | | | | Salt 3 | | | | |

The sensitivity, resolution and LWR in Tables 1 and 2 indicate that the smaller the numerical value, the better the effect. It can be seen that Examples 1 to 10 and Examples 11 to 20 tend to be superior in sensitivity, resolution and LWR to Comparative Examples 1 to 3 and Comparative Examples 4 to 6. It can be seen that, from this result, since the sulfonium salt in one aspect of the present invention has a group selected from the group consisting of —S—, —SO— and —SO₂— at the ortho-position with respect to the sulfonio group (S⁺) and it has the structure having at least one substituent, it is highly sensitive to an electron beam and ArF excimer laser, and tends to efficiently absorb an active energy beam and efficiently generate an acid. It is also found that the resist composition containing the sulfonium salt having the above structure as the acid generator has excellent resolution and tend to reduce LWR in fine patterns.

It can be seen that in Examples 10 and 20, the combination of the sulfonium salts 1 and 10 improves the resolution and the LWR without lowering the sensitivity. From this result, it can be seen that the sulfonium salt according to one aspect of the present invention can also function as a PDB by adjusting the counter anion.

In Examples 1 to 20, the Polymer A or the Polymer B is used as the component (B), but the sulfonium salt according to one aspect of the present invention also tends to be excellent in sensitivity and lithographic properties such as resolution and LWR when used in combination with the component (B) other than the Polymer A or the Polymer B.

### INDUSTRIAL APPLICABILITY

The sulfonium salt according to one aspect of the present invention is useful as an acid generator in a resist composition because it efficiently absorbs an active energy beam and efficiently generates an acid. Further, when the acid generator is used in a resist composition, it tends to have excellent resolution in lithography and can reduce LWR in a fine pattern.

1. A sulfonium salt represented by a following general formula (1), 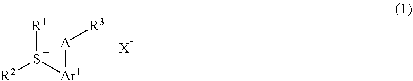
wherein in the general formula (1), each of R¹ to R³ is independently an alkyl group having 1 to 30 carbon atoms, an aryl group having 6 to 30 carbon atoms, or a heteroaryl group having 4 to 30 carbon atoms;
at least one carbon-carbon single bond contained in the alkyl group is optionally substituted with a carbon-carbon double bond or a carbon-carbon triple bond;
at least one methylene group contained in the alkyl group is optionally substituted with at least any divalent heteroatom-containing group selected from a group consisting of —O—, —CO—, —NH—, —S—, —SO—, and —SO₂—;
Ar¹ is an arylene group having 6 to 30 carbon atoms;
at least one of R¹, R², R³ and Ar¹ has at least any substituent (R) selected from a group consisting of a monovalent organic group having 1 to 20 carbon atoms, an amino group, a hydroxyl group, a cyano group, a nitro group, and a halogen atom;
at least two of R¹ to R³, Ar¹, and the substituent (R) optionally form a ring together with the sulfur atom to which R¹, R² or Ar¹ is bonded and/or A, directly with a single bond or via any divalent group selected from a group consisting of an oxygen atom, a sulfur atom, a nitrogen atom-containing group, and an alkylene group;
A is a divalent group selected from a group consisting of —S—, —SO—, and —SO₂—;
Ar¹ is substituted with the A at an ortho-position with respect to the sulfonio group (S⁺); and
X⁻ is an anion.

2. The sulfonium salt of claim 1, wherein the sulfonium salt is represented by a following general formula (2), 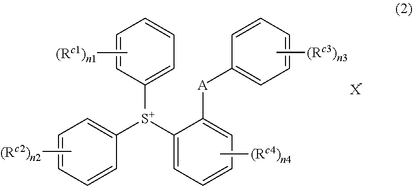
wherein in the general formula (2), each of A and X⁻ is the same as each of A and X⁻ of the general formula (1);
each of R^{c1} to R^{c4} is independently at least one of a monovalent group selected from a group consisting of a monovalent organic group having 1 to 20 carbon atoms, an amino group, a hydroxyl group, a cyano group, a nitro group, and a halogen atom;
at least two selected from a group consisting of benzene rings bonded to the sulfonio group (S⁺); benzene rings bonded to A; R^{c1}; R^{c2}; R^{c3}; and R^{c4}; optionally form a ring together with the sulfonio group (S⁺) bonded to the benzene ring and/or A bonded to the benzene ring, directly with a single bond or via any divalent group selected from a group consisting of an oxygen atom, a sulfur atom, a nitrogen atom-containing group, and an alkylene group;
each of n1 to n3 is an integer of 0 to 5;
n4 is an integer of 0 to 4; and
n1 to n4 satisfy n1+n2+n3+n4>0.

3. The sulfonium salt of claim 1, wherein the anion is at least any selected from a group consisting of a sulfonate anion, a carboxylate anion, an imide anion, and a methide anion.

4. An acid generator comprising the sulfonium salt of claim 1.

5. A resist composition comprising the acid generator of claim 4 and an acid reactive compound.

6. The resist composition of claim 5, wherein the acid reactive compound is an acid dissociative polymer.

7. The resist composition of claim 5, further comprising a water-repellent polymer.

8. A method for manufacturing a device, comprising:
a resist film formation step of applying the resist composition of claim 5 on a substrate to form a resist film;
a photolithography step of exposing the resist film using an active energy beam; and
a patterning step of developing the exposed resist film to obtain a photoresist pattern.

9. The method of manufacturing the device of claim 8, wherein the active energy beam is an electron beam or extreme ultraviolet radiation, EUV.
